SEQUENCE LISTING (2 sequences)

SEQ ID NO: 1 - DNA, 4473 bp, Human
  CDS (175)..(3942)
aaggggaggtaaccctggcccctttggtcggggccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggag

SEQ ID NO: 2 - protein, 1255 aa, Human [sequence omitted]

(19) United States
(12) Patent Application Publication
(10) Pub. No.: US 2012/0171201 A1
(43) Pub. Date: Jul. 5, 2012

(54) METHODS OF TREATING HER2 POSITIVE CANCER WITH HER2 RECEPTOR ANTAGONIST IN COMBINATION WITH MULTI-ARM POLYMERIC CONJUGATES OF 7-ETHYL-10-HYDROXYCAMPTOTHECIN

(75) Inventor: Puja Sapra, Edison, NJ (US)

(73) Assignee: ENZON PHARMACEUTICALS, INC., Bridgewater, NJ (US)

(21) Appl. No.: 13/384,270

(22) PCT Filed: Jul. 21, 2010
(86) PCT No.: PCT/US10/42686

§ 371 Date: Mar. 13, 2012

Related U.S. Application Data
(60) Provisional application No. 61/227,599, filed on Jul. 22, 2009.

(51) Int. Cl. A61K 39/395; A61P 35/00; A61P 35/04; A61K 31/713

(52) U.S. Cl. 424/133.1; 424/130.1; 514/44 A

(57) ABSTRACT

The present invention relates to methods of treating a HER2 positive cancer in mammals. The present invention includes administering a HER2 antagonist in combination with a polymeric prodrug of 7-ethyl-10-hydroxycamptothecin to the mammals in need thereof.

### CROSS-REFERENCE TO RELATED APPLICATION

This application claims the benefit of priority from U.S. Provisional Patent Application Ser. No. 61/227,599, filed Jul. 22, 2009, the contents of which are incorporated herein by reference.

### FIELD OF THE INVENTION

The present invention relates to methods of treating a HER2 positive cancer. In particular, the invention relates to methods of treating a HER2 positive cancer in a mammal by administering a HER2 antagonist in combination with polyethylene glycol conjugates of 7-ethyl- 10-hydroxycamptothecin.

### BACKGROUND OF THE INVENTION

Breast cancer is the most common type of cancer among women in the United States. Recent studies show that approximately 20-25% of breast cancers are HER2 (Human Epidermal Growth Factor Receptor 2) positive. The HER2 protein, also called the HER2 receptor or HER2/neu or ErbB2, is found on the surface of some normal cells in the body. HER2 plays a role in regulating cell growth and survival. HER2 protein, encoding genes and antibodies to HER2 protein are described in detail in U.S. Pat. No. 6,165,464, incorporated by reference herein in its entirety.

Studies show that breast cancer may be more aggressive when the breast cancer tumors over-express the HER2 protein. HER2 positive tumors grow and spread more quickly than tumors that are not HER2 positive. In HER2 positive breast cancer, the cancer cells have an abnormally high of HER2 gene copies per cell. See Slamon D J. et al., Science 244:707-712, 1989; and Pegram M. et al., Semin. Oncol. 27: 13-19. 2000. It has been reported that HER positive breast cancer recurs 2.5 times more than non-HER2 positive cancer. It has also been suggested that HER2 overexpression is associated with resistance to chemotherapeutic agents.

Trastuzumab is a humanized monoclonal antibody which binds selectively to the domain IV of HER2 (or HER2/neu) receptor. Trastuzumab inhibits tumor cell growth by binding to the HER2 protein. Clinical studies showed that the use of trastuzumab reduced the risk of a relapse among those with the aggressive HER2 positive cancer by more than half.

There have been various trials to treat cancer with trastuzumab in combination with chemotherapeutic agents in an attempt to achieve synergistic effects or reduce side effects of therapeutic agents. To name a few, the combination therapy with trastuzumab includes docetaxel/gefitinib/trastuzumab, capecitabine/paclitaxel/trastuzumab, carboplatin/docetaxel/trastuzumab, carboplatin/gemcitabine/paclitaxel/trastuzumab, carboplatin/paclitaxel/trastuzumab, cisplatin/docetaxel/trastuzumab, cyclophosphamide/doxorubicin/trastuzumab, cyclophosphamide/fluorouracil/methotrexate/trastuzumab, etc. See also, for example, U.S. Pat. Nos. 6,313,138; 6,462,017; 6,537,988; and 6,846,816. It has been shown that trastuzumab in combination with chemotherapy extended survival in women both in early stage and late stage metastatic cancer. For example, median survival increased to 26.2 months for patients receiving trastuzumab and chemotherapy, compared with 20.0 months patients receiving chemotherapy alone.

Unfortunately, patients need to receive trastuzumab therapy over a long period of time such as a year. Such long term treatment with trastuzumab has adverse effects. There have been reports that the treatment with trastuzumab alone or in combination with chemotherapy has resulted in heart failure. It is also reported that it is dangerous for patients to receive trastuzumab in combination with anthracycline-based chemotherapy. A significant of patients receiving trastuzumab in combination with chemotherapeutic agents such as doxorubicin, cyclophosphamide, and either paclitaxel or docetaxel developed heart failure. As such, trastuzumab-associated therapy requires patients to have their heart function test prior to and during trastuzumab-associated therapy and it is recommended that patients with heart problems not receive or stop trastuzumab-associated therapy. The prolonged use of tratuzumab may also worsen chemotherapy-induced neutropenia.

Thus, there continues to be a need for methods for treating a HER2 positive cancer. The present invention addresses this need.

### SUMMARY OF THE INVENTION

In one aspect of the present invention, there is provided a method of treating a HER2 positive cancer in a mammal. The method includes administering a HER2 receptor antagonist to the mammal in combination with an effective amount of a compound of Formula (I):

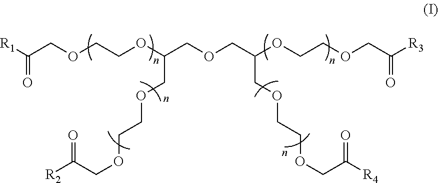

wherein

R₁, R₂, R₃ and R₄ are independently OH or

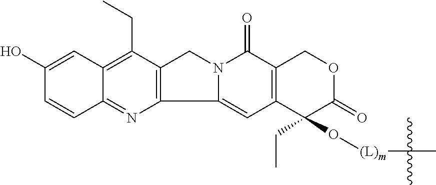

wherein

L is a bifunctional linker, and each L is the same or different, when (m) is equal to or greater than 2;

(m) is 0 or a positive integer; and

(n) is a positive integer;

provided that R₁, R₂, R₃ and R₄ are not all OH; or a pharmaceutically acceptable salt thereof to the mammal.

In an alternative aspect, there is provided a method of treating a HER2 positive cancer in a mammal. The method includes administering to said mammal a HER2 receptor antagonist in combination with an effective amount of a camptothecin, a camptothecin analog, a polymeric conjugate of a camptothecin or analog thereof, or a pharmaceutically acceptable salt thereof.

In one embodiment, the method is conducted by administering a HER2 receptor antagonist plus a polymeric conjugate of a camptothecin or analog thereof to a mammal having a HER2 positive cancer. The polymeric conjugate includes a compound of Formula (II) or (III):

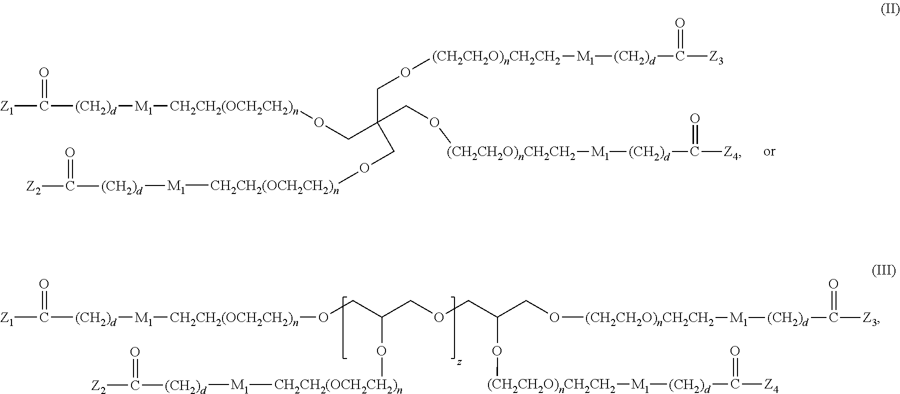

wherein

Z₁, Z₂, Z₃ and Z₄ are independently OH or (L)ₘ-D;

L is a bifunctional linker;

D is a camptothecin or a camptothecin analog;

M₁ is O, S, or NH;

(d) is zero or a positive integer of from about 1 to about 10;

(z) is zero or a positive integer of from 1 to about 29;

(m) is 0 or a positive integer, wherein each L is the same or different when (m) is equal to or greater than 2; and

(n) is a positive integer of from about 10 to about 2,300 so that the polymeric portion of the compound has the total average molecular weight of from about 2,000 to about 100,000 daltons,

provided that Z₁, Z₂, Z₃ and Z₄ are not all OH.

In one embodiment, the HER2 antagonist employed in the methods described herein includes trastuzumab marketed under the trademark Herceptin®, as described in detail by U.S. Pat. Nos. 5,821,337 and 6,165,464, incorporated by reference herein.

In one preferred embodiment, the polymeric prodrugs of 7-ethyl-10-hydroxycamptothecin employed in the methods described herein include four-arm PEG-7-ethyl-10-hydroxycamptothecin conjugates having the structure of

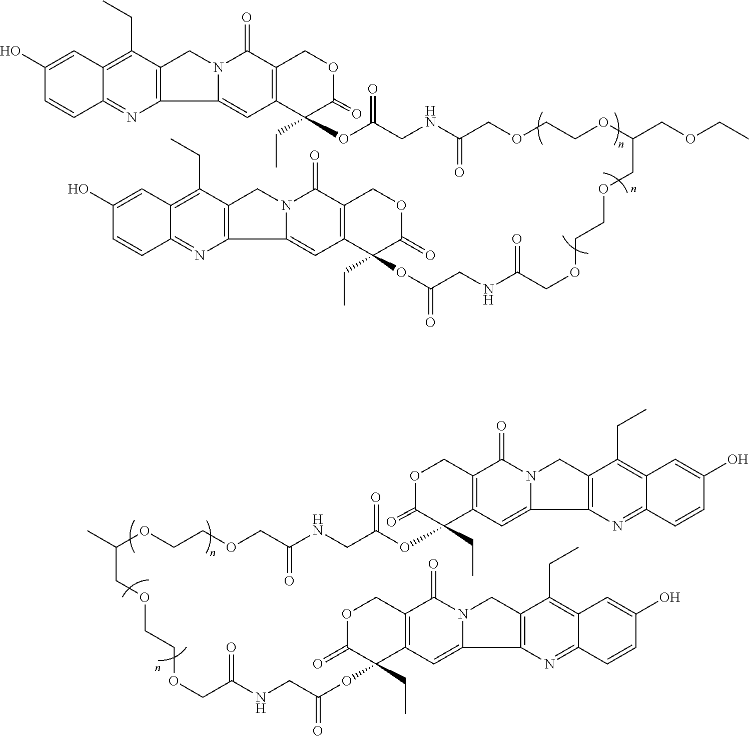

wherein (n) is from about 28 to about 341, preferably from about 114 to about 239, and more preferably about 227.

In yet another embodiment, the method described herein includes:

(a) determining the presence of HER2 positive cancer in a mammal having a cancer; and

(b) administering an effective amount of a HER2 receptor antagonist in combination with an effective amount of a compound of Formula (I) (or Formula (II) or (III)) to a mammal having a HER2 positive cancer.

In another aspect, the present invention provides a method of increasing HER2 receptor antagonist effects in a mammal having a HER2 positive cancer.

In yet another aspect, the present invention provides a method of inhibiting the growth or proliferation of HER2 positive cells, as well as a method of delivering a camptothecin such as 7-ethyl-10-hydroxycomptothecin to a HER2 positive cell in a mammal.

One advantage of the present invention is that the present invention provides a means to utilize HER2 antagonist-based therapy effectively for the treatment of patients who did not respond to HER2 antagonist-containing therapy, or patients who initially responded but later developed resistance to a HER2 antagonist. Patients can benefit from unexpected lack and/or reduction in resistance to a HER2 antagonist such as trastuzumab and pertuzumab.

Another advantage is that the present invention provides a means to treat patients with poor prognosis. HER2 is considered to be correlated with drug resistance and overall poor prognosis. A HER2 antagonist, when administered with the compounds of Formula (I) (alternatively compounds of Formula (II) or (III)) described herein according to the present invention is significantly effective in inhibiting tumor growth and/or proliferation, compared to treatments in which a HER2 antagonist is not administered in combination with the compounds described herein.

Yet another advantage is that the present invention increases the therapeutic efficacy of a HER2 antagonist, and allows certain patients in need to receive HER2-associated therapy for a lesser period or amount, when compared to HER2 therapy alone. Any side-effects associated with or which result from HER2-associated therapy can be alleviated by the enhanced efficacy of HER2 antagonist therapy.

Further advantages will be apparent from the following description and drawings.

For purposes of the present invention, “HER2 positive cancer” and “HER2 over-expressing cancer” are used interchangeably. In HER2-positive cancer cells, there is an excess amount of the HER2 protein on the cell surface and/or amplification of the encoding HER2/neu gene. Levels of HER2 expression can be measured by techniques known in the art, as well as those methods described later. HER2 positive cancer has greater expression of the HER2 protein or gene, as compared to non-HER2 positive cancer or normal cells or tissues. For example, HER2 is determined by immunohistochemical (“IHC”) assays that measure the amount of HER2 protein expressed on the surface of cancer cells. IHC assays are scored on a scale of 0 to 3+ based on the staining intensity and completeness of cell membrane staining. For example, a cancer that scores 3+ on an IHC assay is considered to be HER2 positive cancer. A cancer that scores 2+ on an IHC assay may be further tested by a fluorescence in-situ hybridization (“FISH”) assay, where a positive FISH assay confirms that the cancer is HER2 positive. A FISH assay measures the of HER2/neu gene copies present in cancer cells. FISH test results are provided by the ratio of the of HER2 signals to the of chromosome 17 signals among 20 interphase nuclei in tumor cells. Normal specimens show a ratio of <2.0, while specimens with amplification of HER2/neu have a ratio of greater than or equal to 2.0 and are defined as HER2-positive (FISH +).

The terms “HER2 receptor antagonist” and “HER2 antagonist” refer to compounds which inhibit expression or function of the HER2 protein or gene. For purposes of the present invention, a HER2 antagonist refers to, e.g., receptor tyrosine kinase inhibitors, especially HER2 receptor protein inhibitors. Simply by way of example, HER2 antagonists include anti-HER2 antibodies. The definition of HER2 antagonists is also intended to include antisense HER2 oligonucleotides.

For purposes of the present invention, the term “adjuvant treatment” refers to treatment given in addition to the primary (initial) treatment. Adjuvant treatment is an additional treatment designed to help reach the ultimate goal.

For purposes of the present invention, the term “early” or “early-stage” breast cancer means that the cancer has not spread beyond the breast or lymph nodes under the arm (known as axillary lymph nodes). Stage 0, I, and II, as well as some stage III cancers, are usually considered early-stage.

For purposes of the present invention, refractory or resistant cancers are defined as cancers that have not responded to previous anticancer therapy or treatment which does not include the compounds of Formula (I) (alternatively, compounds of Formula (II) or (III)) described herein. In one aspect, the cancers are resistant or refractory to a HER2 receptor antagonist such as HER2 antibodies (e.g. trastuzumab and pertuzumab) when used alone or in combination with chemotherapy which does not include the compound of Formula (I) (alternatively, compounds of Formula (II) or (III)). In one embodiment, the cancers are refractory or resistant to Herceptin® treatment alone, or to Herceptin® plus chemotherapy which does not include the compounds of Formula (I) described herein. The cancers can be refractory or resistant at the beginning of treatment, or they may become refractory or resistant during/after treatment. Thus, refractory cancers include tumors that do not respond at the onset of treatment or respond initially for a short period but fail to respond to treatment. Refractory cancers also include tumors that respond to treatment with anticancer therapy but fail to respond to subsequent rounds of therapies. For the purposes of this invention, refractory cancers can also encompass tumors that appear to be inhibited by treatment with anticancer therapy but recur up to five years, sometimes up to ten years or longer, after treatment is discontinued. The anticancer therapy can employ chemotherapeutic agents alone, radiation alone or combinations thereof For ease of description and not limitation, it will be understood that the refractory cancers are interchangeable with resistant cancers.

For purposes of the present invention, successful treatment of a refractory or resistant cancer shall be understood to mean that refractory or resistant symptoms or conditions are inhibited, minimized or attenuated during and/or after the combination treatment described herein, when compared to that observed in the absence of the combination treatment described herein. The minimized, attenuated or inhibited refractory conditions can be confirmed by clinical markers contemplated by the artisan in the field. In one example, successful treatment of refractory or resistant cancer shall be deemed to occur when at least 5% or preferably 10%, more preferably 20% or higher (i.e., 30, 40, 50% or more) inhibition or decrease in tumor growth and/or recurrence including other clinical markers contemplated by the artisan in the field is realized when compared to that observed in the absence of the treatment described herein. Clinical markers which show changes in the severity and magnitude of the refractory cancers can be determined by clinicians.

For purposes of the present invention, the terms “cancer” and “tumor” are used interchangeably, unless otherwise indicated. Cancer encompasses benign, malignant and/or metastatic cancer, unless otherwise indicated. Cancers may be more aggressive or less aggressive. The aggressive phenotype refers to the proliferation rate and the ability to form tumors and metastasize. Aggressive cancers proliferate more quickly, and form tumors and metastasize more easily, as compared to less-aggressive tumors.

For purposes of the present invention, “treatment of tumor/cancer” shall be understood to mean inhibition, reduction, or amelioration of tumor growth, tumor burden and metastasis, remission of tumor, or inhibition of recurrences of tumor and/or neoplastic growths realized in patients after completion of the combination therapy described herein, as compared to patients who have not received the combination treatment described herein. Successful treatment is deemed to occur when a patient achieves positive clinical results. For example, successful treatment of a tumor shall be deemed to occur when at least 10% or preferably 20%, more preferably 30% or higher (i.e., 40%, 50%) decrease in tumor growth including other clinical markers contemplated by the artisan in the field is realized when compared to that observed in the absence of the combination treatment described herein. Other methods for determining changes in a tumor clinical status resulting from the treatment described herein include: biopsies such as a tumor biopsy, an immunohistochemistry study using antibody, radioisotope, dye, and complete blood count (CBC).

For purposes of the present invention, diseases or disorders associated with HER2 over-expression contemplated according to the present invention include conditions in which the HER2 protein or gene plays a role in the pathology or progression of the condition.

The terms “effective amounts” and “sufficient amounts” for purposes of the present invention shall mean an amount which achieves a desired effect or therapeutic effect as such effect is understood by those of ordinary skill in the art. An effective amount for each mammal or human patient to be treated is readily determined by the artisan in a range that provides a desired clinical response while avoiding undesirable effects that are inconsistent with good practice. Dose ranges are provided hereinbelow.

For purposes of the present invention, the term “residue” shall be understood to mean that portion of a compound, to which it refers, e.g., 7-ethyl-10-hydroxycamptothecin, amino acid, etc. that remains after it has undergone a substitution reaction with another compound.

For purposes of the present invention, the term “polymeric residue” or “PEG residue” shall each be understood to mean that portion of the polymer or PEG which remains after it has undergone a reaction with, e.g., an amino acid, 7-ethyl-10-hydroxycamptothecin-containing compounds.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆hydrocarbonyl, aryl, and amino groups.

For purposes of the present invention, the telin “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propynyl (i.e., propargyl), and 3-hexynyl.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “halogen” or “halo” refers to fluorine, chlorine, bromine, and iodine.

For purposes of the present invention, the term “heteroatom” refers to nitrogen, oxygen, and sulfur.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl.

For purposes of the present invention, “positive integer” shall be understood to include an integer equal to or greater than 1 (e.g., an integer from about 1 to about 10, from about 1 to about 6) and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

For purposes of the present invention, the terms, “nucleic acid” or “nucleotide” apply to deoxyribonucleic acid (“DNA”), ribonucleic acid, (“RNA”) whether single-stranded or double-stranded, unless otherwise specified, and any chemical modifications thereof.

Preferably, a mammal to be treated according to the invention is a human.

### BRIEF DESCRIPTION OF DRAWINGS

FIG. 1 illustrates the stability of 4 arm-PEG-Gly-(7-ethyl-10-hydroxycamptothecin) as described in Example 4, in human plasma, phosphate buffer solution, and saline.

FIG. 2 illustrates the effect of pH on stability of 4 arm-PEG-Gly-(7-ethyl-10-hydroxy-camptothecin) as described in Example 4.

FIG. 3A illustrates pharmacokinetic profiles of 4 arm-PEG-Gly-(7-ethyl-10-hydroxy-camptothecin) as described in Example 5.

FIG. 3B illustrates pharmacokinetic profiles of 4 arm-PEG-Gly-(7-ethyl-10-hydroxycamptothecin) as described in Example 5. Enterohepatic circulation of 4 arm-PEG-Gly-(7-ethyl-10-hydroxycamptothecin) conjugates is indicated.

FIG. 4 illustrates the inhibition in tumor growth in mice xenografted with human JIMT-1 breast tumor that is refractory to Herceptin® and pertuzumab, as described in Example 6.

FIG. 5 illustrates the inhibition in tumor growth in mice xenografted with human N87 gastric cancer, as described in Example 7.

### DETAILED DESCRIPTION OF THE INVENTION

### A. Overview

In one aspect of the invention, there are provided methods of treating a HER2 positive cancer in a mammal. The method includes:

administering a HER2 receptor antagonist in combination with an effective amount of a compound of Formula (I):

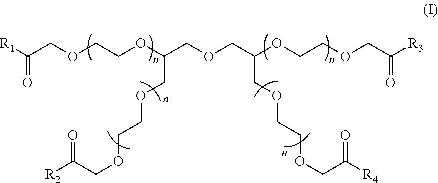

wherein

R₁, R₂, R₃ and R₄ are independently OH or

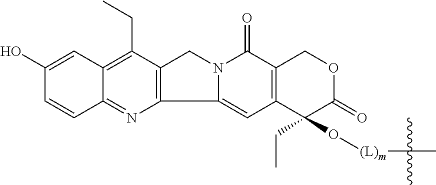

wherein

L is a bifunctional linker;

(m) is 0 or a positive integer, preferably zero or an integer from about 1 to about 10 (e.g., 1, 2, 3, 4, 5, 6), wherein each L is the same or different when (m) is equal to or greater than 2; and

(n) is a positive integer;

provided that R₁, R₂, R₃ and R₄ are not all OH; or a pharmaceutically acceptable salt thereof, to said mammal

In one preferred embodiment, the method includes administering, to the mammal, a HER2 receptor antagonist in combination with a compound of Formula (I) in which R₁, R₂, R₃ and R₄ are all:

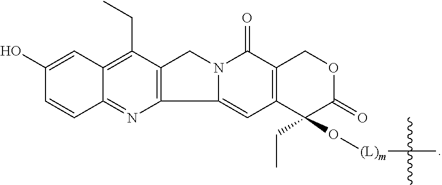

In more preferred aspect, the method includes administering a HER2 receptor antagonist in combination with a compound of Formula (Ia):

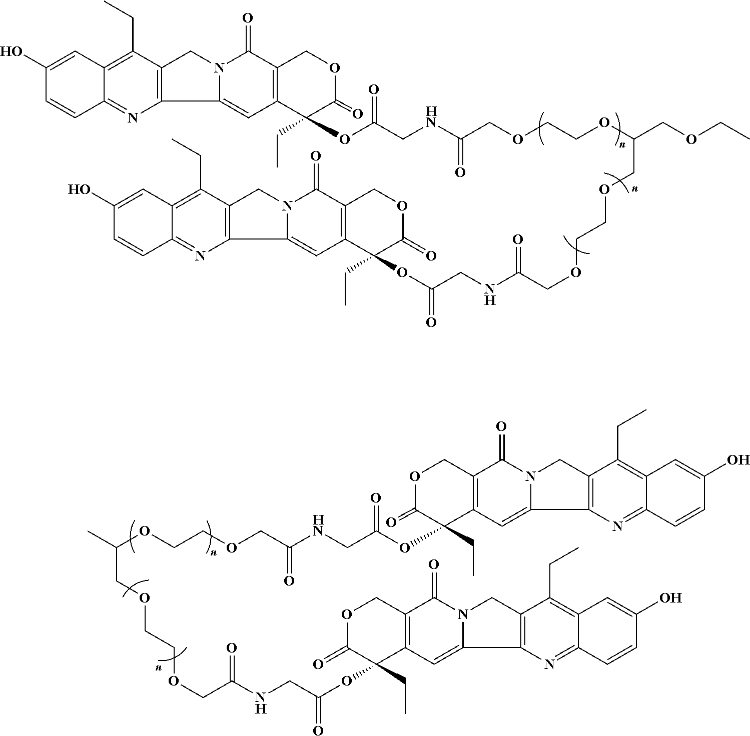

wherein (n) is about 227 so that the polymeric portion of the compound has the total average molecular weight of about 40,000 daltons.

In an alternative aspect, there are provided methods of treating a HER2 positive cancer in a mammal. The method includes administering to said mammal a HER2 receptor antagonist in combination with an effective amount of a camptothecin, a camptothecin analog, a polymeric conjugate of a camptothecin or analog thereof, or a pharmaceutically acceptable salt thereof.

In one embodiment, the method is conducted by administering a HER2 receptor antagonist plus a polymeric conjugate of a camptothecin or analog thereof to a mammal having a HER2 positive cancer. The polymeric conjugate includes a compound of Formula (II) or (III):

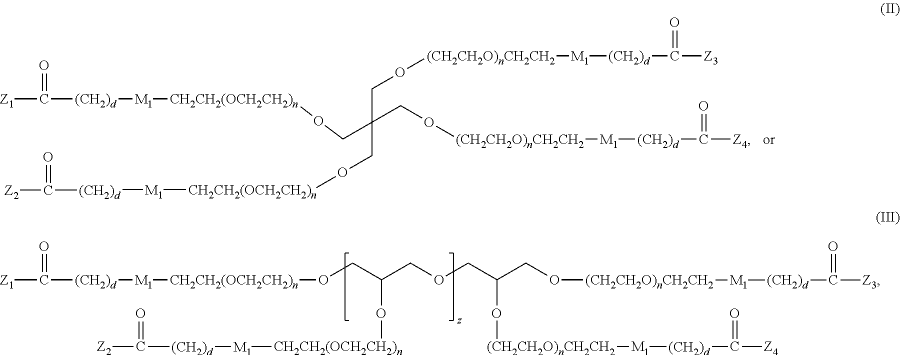

wherein

Z₁, Z₂, Z₃ and Z₄ are independently OH or (L)ₘ-D;

L is a bifunctional linker;

D is a camptothecin or a camptothecin analog;

M₁ is O, S, or NH, preferably O;

(d) is zero or a positive integer of from about 1 to about 10, preferably, 0, 1, 2, or 3, and more preferably, 0 or 1;

(z) is zero or a positive integer of from 1 to about 29, preferably, 1, 5, 13 or 29;

(m) is 0 or a positive integer, preferably zero or an integer from about 1 to about 10 (e.g., 1, 2, 3, 4, 5, 6), wherein each L is the same or different when (in) is equal to or greater than 2; and

(n) is a positive integer of from about 10 to about 2,300 so that the polymeric portion of the compound has the total average molecular weight of from about 2,000 to about 100,000 daltons,

provided that Z₁, Z₂, Z₃ and Z₄ are not all OH.

In one particular embodiment, SN38 is attached at its 20-OH position to the multi-armed polyethylene glycol of Formula (II) or (III) via the bifunctional linker such as glycine, alanine, methionine, etc. Alternatively, camptothecin, topotecan or CPT-11 is attached at its 20-OH position to the multi-armed polyethylene glycol of Formula (II) or (III) via the bifunctional linker such as glycine, alanine, methionine, sarcosine, etc.

The HER2 antagonist and the compound of Formula (I) (alternatively, compounds of Formula (II) or (III)) are administered in amounts which are sufficient to achieve a desired therapeutic effect.

The HER2 positive cancers which can be treated with the methods described herein include, but are not limited to, solid tumors, breast cancer, gastric cancer, ovarian cancer, stomach cancer, uterine cancer, uterine serous endometrial carcinoma, prostate cancer, bladder cancer, salivary gland carcinoma, renal adenocarcinoma, and mammary gland carcinoma. The forgoing list is not meant to be exclusive and those of ordinary skill will realize that other HER2 cancers not specifically mentioned herein are intended for inclusion.

The HER2 positive cancer can be metastatic or non-metastatic.

In one aspect, the methods described herein can be useful in the treatment of a HER2 positive cancer which is resistant or refractory to a HER2 receptor antagonist such as trastuzumab and pertuzumab when used alone or in combination with chemotherapy which does not include the compound of Formula (I) (alternatively, compounds of Formula (II) or (III)). The combination treatment described herein is also useful for the treatment of a HER2 positive cancer which is sensitive to the anti-HER2 antibodies. Without being bound by any theory, the methods described herein enhance therapeutic efficacy of HER2 antagonist and/or alleviate resistance to the HER2 receptor antagonists by HER2 positive cancer, when compared to the treatment with the anti-HER2 antibodies without the compound of Formula (I) described herein.

In a further aspect, the method described herein includes a step of identifying a patient with a HER2 positive cancer.

In an alternate aspect, the present invention provides a method of treating a disease or disorder associated with higher levels of the HER2 protein or gene (e.g., gene expression), compared to that observed in a mammal with normal expression of HER2 (or without excessive expression of HER2). Pathological conditions which involve excessive expression of the HER2 protein or gene benefit from the treatment described herein. The method can be conducted wherein a HER2 receptor antagonist is administered in combination with the compound of Formula (I) (alternatively, compounds of Formula (II) or (III)) or pharmaceutically acceptable salt thereof.

In one embodiment, a HER2 receptor antagonist can be administered with the compound of Formula (I) (alternatively, compounds of Formula (II) or (III)) concurrently or sequentially.

In another embodiment, the HER2 receptor antagonist includes anti-HER2 antibodies, antisense ErbB2 oligonucleotides, and combinations thereof.

In one preferred embodiment, the method includes the steps of:

(a) identifying a mammal having a HER2 positive cancer by e.g., determining the presence, in the mammal, of a cancer that overexpresses HER2; and

(b) administering an effective amount of a HER2 receptor antagonist, preferably trastuzumab, in combination with an effective amount of a compound of Formula (Ia):

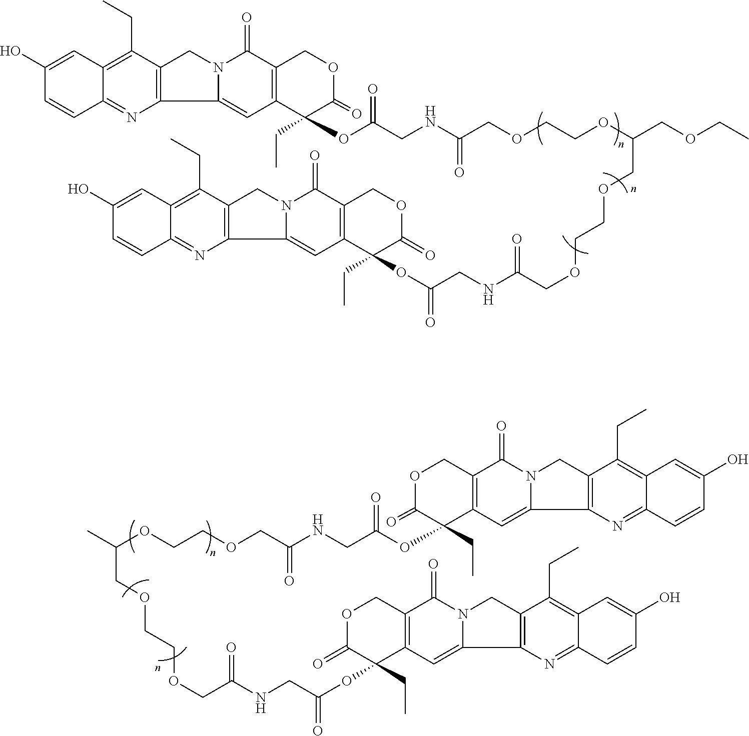

or a pharmaceutically acceptable salt thereof to the mammal having a HER2 positive cancer,

wherein (n) is preferably about 227 so that the total molecular weight of the polymeric portion of the compound of Formula (Ia) is about 40,000 daltons.

In an alternate aspect, there are provided methods of treating a tyrosine kinase-dependent disease or disorder in a mammal. The HER2 receptor is a tyrosine kinase and it is implicated in a pathological condition such as cancer. The method includes administering a HER2 receptor antagonist in combination with a compound of Formula (I) (or Formula (II) or (III)) to a mammal having an overexpressing HER2-dependent disease. These methods preferably include the step of identifying a patient having such a disease or disorder.

In yet another aspect, the present invention provides a method of increasing HER2 receptor antagonist effects in a mammal having a HER2 positive cancer. The method includes administering a HER2 receptor antagonist in combination with an effective amount of a compound of Formula (I) (alternatively, compounds of Formula (II) or (III)).

In yet another aspect, the present invention provides a method of inhibiting the growth, proliferation, or metastasis of HER2 positive cells in a mammal by administering a HER2 receptor antagonist in combination with the compound of Formula (I) (alternatively, compounds of Formula (II) or (III)) described herein or pharmaceutically acceptable salt thereof to a mammal or by contacting a HER2 receptor antagonist in combination with the compound of Formula (I) (alternatively, compounds of Formula (II) or (III)) described herein or pharmaceutically acceptable salt thereof with cancer cells or tissues. In one particular embodiment, the method includes:

(a) determining the presence of a HER2 expression in cells; and

(b) administering a HER2 receptor antagonist and an effective amount of a compound of Formula (I) (or Formula (II) or (III)) of claim 1 or a pharmaceutically acceptable salt thereof to a mammal in need thereof. In certain aspects, the cells are cancerous cells.

### B. Polymeric Compounds

1. Multi-Arm Polymers

The polymeric portion of the compounds described herein includes multi-armed PEG's attached to 20-OH group of 7-ethyl-10-hydroxycamptothecin. In one aspect of the present invention, the polymeric prodrugs of 7-ethyl-10-hydroxycamptothecin include four-arm PEG, prior to conjugation, having the following structure of

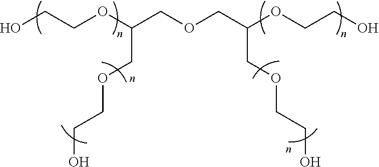

wherein (n) is a positive integer.

Alternatively, the polymeric compounds employ four-arm PEG, prior to conjugation, having the structure:

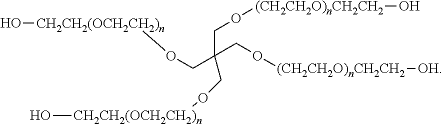

The multi-armed PEG's are those described in NOF Corp. Drug Delivery System catalog, Ver. 8, April 2006, the disclosure of which is incorporated herein by reference.

In one preferred embodiment of the invention, the degree of polymerization for the polymer (n) is from about 28 to about 341 to provide polymers having the total average molecular weight of from about 5,000 Da to about 60,000 Da, and preferably from about 114 to about 239 to provide polymers having the total average molecular weight of from about 20,000 Da to about 42,000 Da. (n) represents the of repeating units in the polymer chain and is dependent on the molecular weight of the polymer. In one particularly preferred embodiment of the invention, (n) is about 227 to provide the polymeric portion having the total average molecular weight of about 40,000 Da.

2. Bifunctional Linkers

In certain preferred aspects of the present invention, bifunctional linkers include an amino acid. The amino acid which can be selected from any of the known naturally-occurring L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name but a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L), hydrophobic or non-hydrophobic, are also contemplated to be within the scope of the invention. Simply by way of example, amino acid analogs and derivates include:
2-aminoadipic acid, 3-aminoadipic acid, beta-alanine, beta-aminopropionic acid,
2-aminobutyric acid, 4-aminobutyric acid, piperidinic acid, 6-aminocaproic acid,
2-aminoheptanoic acid, 2-aminoisobutyric acid, 3-aminoisobutyric acid,
2-aminopimelic acid, 2,4-aminobutyric acid, desmosine, 2,2-diaminopimelic acid,
2,3-diaminopropionic acid, N-ethylglycine, N-ethylasparagine, 3-hydroxyproline,
4-hydroxyproline, isodesmosine, allo-isoleucine, N-methylglycine or sarcosine,
N-methylisoleucine, 6-N-methyllysine, N-methylvaline, norvaline, norleucine, ornithine, and others too numerous to mention, that are listed in 63 Fed. Reg., 29620, 29622, incorporated by reference herein. Some preferred L groups include glycine, alanine, methionine or sarcosine. For example, the compounds can be among:

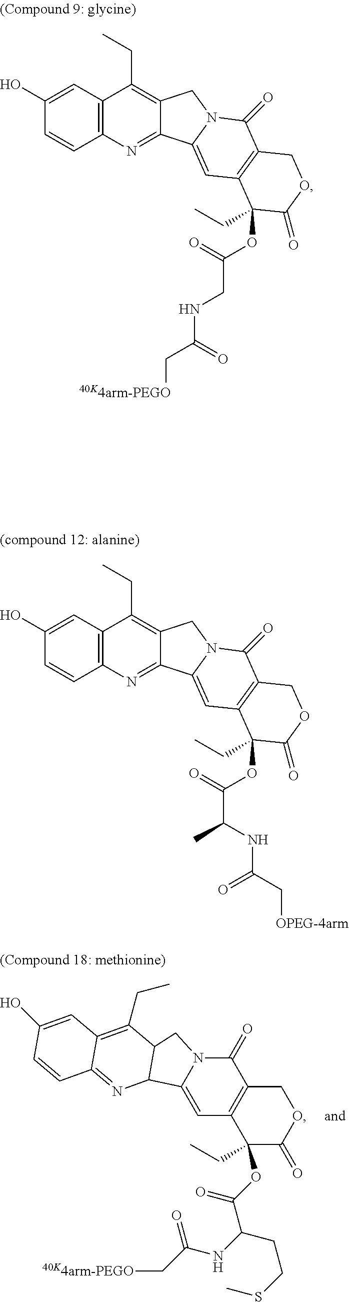

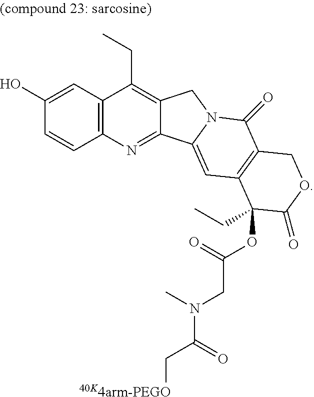

For ease of the description and not limitation, only one arm of the four-arm PEG is shown. One arm, up to four arms of the four-arm PEG can be conjugated with 7-ethyl-10-hydroxy-camptothecin.

More preferably, the treatment described herein employs compounds including a glycine as the linker group (L).

In an alternative aspect of the present invention, L after attachment between the polymer and 7-ethyl-10-hydroxycamptothecin can be selected among:

—[C(═O)]ᵥ(CR₂₂R₂₃)ₜ—,

—[C(═O)]ᵥ(CR₂₂R₂₃)ₜ—O—,

—[C(═O)]ᵥ(CR₂₂R₂₃)ₜ—NR₂₆—,

—[C(═O)]ᵥ(CR₂₂R₂₃)ₜ—,

—[C(═O)]ᵥO(CR₂₂R₂₃)ₜO—,

—[C(═O)]ᵥO(CR₂₂R₂₃)ₜNR₂₆—,

—[C(═O)]ᵥNR₂₁(CR₂₂R₂₃)ₜ—,

—[C(═O)]ᵥNR₂₁(CR₂₂R₂₃)ₜO—,

—[C(═O)]ᵥNR₂₁(CR₂₂R₂₃)ₜNR₂₆—,

—[C(═O)]ᵥ(CR₂₂R₂₃O)ₜ—,

—[C(═O)]ᵥO(CR₂₂R₂₃O)ₜ—,

—[C(═O)]ᵥNR₂₁(CR₂₂R₂₃O)ₜ—,

—[C(═O)]ᵥ(CR₂₂R₂₃O)ₜ(CR₂₄R₂₅)_{y}—,

—[C(═O)]ᵥO(CR₂₂R₂₃O)ₜ(CR₂₄R₂₅)_{y}—,

—[C(═O)]ᵥNR₂₁(CR₂₂R₂₃O)ₜ(CR₂₄R₂₅)_{y}—,

—[C(═O)]ᵥ(CR₂₂R₂₃O)ₜ(CR₂₄R₂₅)_{y}O—,

—[C(═O)]ᵥ(CR₂₂R₂₃)ₜ(CR₂₄R₂₅O)_{y}O—,

—[C(═O)]ᵥO(CR₂₂R₂₃O))ₜ(CR₂₄R₂₅)_{y}O—,

—[C(═O)]ᵥO(CR₂₂R₂₃)ₜ(CR₂₄R₂₅O)_{y}—,

—[C(═O)]ᵥNR₂₁(CR₂₂R₂₃O)ₜ(CR₂₄R₂₅O)_{y}—,

—[C(═O)]ᵥNR₂₁(CR₂₂R₂₃)ₜ(CR₂₄R₂₅O)_{y}—,

—[C(═O)]ᵥ(CR₂₂R₂₃)ₜO—(CR₂₈R₂₉)_{t′}—,

—[C(═O)]ᵥ(CR₂₂R₂₃)ₜNR₂₆—(CR₂₈R₂₉)_{t′}—,

—[C(═O)]ᵥ(CR₂₂R₂₃)ₜS—(CR₂₈R₂₉)_{t′}—,

—[C(═O)]ᵥO(CR₂₂R₂₃)ₜO—(CR₂₈R₂₉)_{t′}—,

—[C(═O)]ᵥO(CR₂₂R₂₃)ₜNR₂₆—(CR₂₈R₂₉)_{t′}—,

—[C(═O)]ᵥO(CR₂₂R₂₃)ₜS—(CR₂₈R₂₉)_{t′}—,

—[C(═O]ᵥNR₂₁(CR₂₂R₂₃)ₜO—(CR₂₈R₂₉)_{t′}—,

—[C(═O]ᵥNR₂₁(CR₂₂R₂₃)ₜNR₂₆—(CR₂₈R₂₉)_{t′}—,

—[C(═O)]ᵥNR₂₁(CR₂₂R₂₃)ₜS—(CR₂₈R₂₉)_{t′}—,

—[C(═O)]ᵥ(CR₂₂R₂₃CR₂₈R₂₉O)ₜNR₂₆—,

—[C(═O)]ᵥ(CR₂₂R₂₃CR₂₈R₂₉O)ₜ—,

—[C(═O)]ᵥO(CR₂₂R₂₃CR₂₈R₂₉O)ₜNR₂₆—,

—[C(═O)]ᵥO(CR₂₂R₂₃CR₂₈R₂₉O)ₜ—,

—[C(═O)]ᵥNR₂₁(CR₂₂R₂₃CR₂₈R₂₉O)ₜNR₂₆—,

—[C(═O)]ᵥNR₂₁(CR₂₂R₂₃CR₂₈R₂₉O)ₜ—,

—[C(═O)]ᵥ(CR₂₂R₂₃CR₂₈R₂₉O)ₜ(CR₂₄R₂₅)_{y}—,

—[C(═O)]ᵥO(CR₂₂R₂₃CR₂₈R₂₉O)ₜ(CR₂₄R₂₅)_{y}—,

—[C(═O)]ᵥNR₂₁(CR₂₂R₂₃CR₂₈R₂₉O)ₜ(CR₂₄R₂₅)_{y}—,

—[C(═O)]ᵥ(CR₂₂R₂₃CR₂₈R₂₉O)ₜ(CR₂₄R₂₅)_{y}O—,

—[C(═O)]ᵥ(CR₂₂R₂₃)ₜ(CR₂₄R₂₅CR₂₈R₂₉O)_{y}—,

—[C(═O)]ᵥ(CR₂₂R₂₃)ₜ(CR₂₄R₂₅CR₂₈R₂₉O)_{y}NR₂₆—,

—[C(═O)]ᵥO(CR₂₂R₂₃CR₂₈R₂₉O)ₜ(CR₂₄R₂₅)_{y}O—,

—[C(═O)]ᵥO(CR₂₂R₂₃)ₜ(CR₂₄R₂₅CR₂₈R₂₉O)_{y}—,

—[C(═O)]ᵥO(CR₂₂R₂₃)ₜ(CR₂₄CR₂₅CR₂₈R₂₉O)_{y}NR₂₆—,

—[C(═O)]ᵥNR₂₁(CR₂₂R₂₃CR₂₈R₂₉O)ₜ(CR₂₄R₂₅)_{y}O—,

—[C(═O)]ᵥNR₂₁(CR₂₂R₂₃)ₜ(CR₂₄R₂₅CR₂₈R₂₉O)_{y}—,

—[C(═O)]ᵥNR₂₁(CR₂₂R₂₃)ₜ(CR₂₄R₂₅CR₂₈R₂₉O)_{y}NR₂₆—,

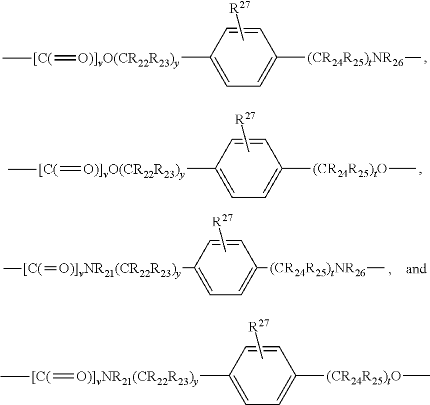

wherein:

R₂₁-R₂₉ are independently selected among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;

(t), (t′) and (y) are independently chosen from zero or a positive integer, preferably from about 1 to about 10 such as 1, 2, 3, 4, 5 and 6; and

(v) is 0 or 1.

The bifunctional linkers contemplated within the scope of the present invention include those in which combinations of substituents and variables are permissible so that such combinations result in stable compounds.

In some preferred embodiments, L can include:

—[C(═O)]ᵥ(CH₂)ₜ—,

—[C(═O)]ᵥ(CH₂)ₜ—O—,

—[C(═O)]ᵥ(CH₂)ₜ—NH—,

—[C(═O)]vO(CH₂)ₜ—,

—[C(═O)]ᵥO(CH₂)ₜO—,

—[C(═O)]ᵥO(CH₂)ₜNH—,

—[C(═O)]ᵥNH(CH₂)ₜ—,

—[C(═O)]ᵥNH(CH₂)ₜO—,

—[C(═O)]ᵥNH(CH₂)ₜNH—,

—[C(═O)]ᵥ(CH₂O)ₜ—,

—[C(═O)]ᵥO(CH₂O)ₜ—,

—[C(═O)]ᵥNH(CH₂O)ₜ—,

—[C(═O)]ᵥ(CH₂O)ₜ(CH₂)_{y}—,

—[C(═O)]ᵥO(CH₂O)ₜ(CH₂)_{y}—,

—[C(═O)]ᵥNH(CH₂O)ₜ(CH₂)_{y}—,

—[C(═O)]ᵥ(CH₂)ₜ(CH₂)_{y}O—,

—[C(═O)]ᵥ(CH₂)ₜ(CH₂O)_{y}—,

—[C(═O)]ᵥO(CH₂O)ₜ(CH₂)_{y}O—,

—[C(═O)]ᵥO(CH₂)ₜ(CH₂O)_{y}—,

—[C(═O)]ᵥNH(CH₂O)ₜ(CH₂)_{y}O—,

—[C(═O)]ᵥNH(CH₂)ₜ(CH₂O)_{y}—,

—[C(═O)]ᵥ(CH₂)ₜO—(CH₂)ₜ′—,

—[C(═O)]ᵥ(CH₂)ₜNH—(CH₂)ₜ′—,

—[C(═O)]ᵥ(CH₂)ₜS—(CH₂)ₜ′—,

—[C(═O)]ᵥO(CH₂)ₜO—(CH₂)ₜ′—,

—[C(═O)]ᵥO(CH₂)ₜNH—(CH₂)ₜ′—,

—[C(═O)]ᵥO(CH₂)ₜS—(CH₂)ₜ′—,

—[C(═O)]ᵥNH(CH₂)ₜO—(CH₂)ₜ′—,

—[C(═O)]ᵥNH(CH₂)ₜNH—(CH₂)ₜ′—,

—[C(═O)]ᵥNH(CH₂)ₜS—(CH₂)ₜ′—,

—[C(═O)]ᵥ(CH₂CH₂O)ₜNH—,

—[C(═O)]ᵥ(CH₂CH₂O)ₜ—,

—[C(═O)]ᵥO(CH₂CH₂O)ₜNH—,

—[C(═O)]ᵥO(CH₂CH₂O)ₜ—,

—[C(═O)]ᵥNH(CH₂CH₂O)ₜNH—,

—[C(═O)]ᵥNH(CH₂CH₂O)ₜ—,

—[C(═O)]ᵥ(CH₂CH₂O)ₜ(CH₂)_{y}—,

—[C(═O)]ᵥO(CH₂CH₂O)ₜ(CH₂)_{y}—,

—[C(═O)]ᵥNH(CH₂CH₂O)ₜ(CH₂)_{y}—,

—[C(═O)]ᵥ(CH₂CH₂O)ₜ(CH₂)_{y}O—,

—[C(═O)]ᵥ(CH₂)ₜ(CH₂CH₂O)_{y}—,

—[C(═O)]ᵥ(CH₂)ₜ(CH₂CH₂O)_{y}NH—,

—[C(═O)]ᵥO(CH₂CH₂O)ₜ(CH₂)_{y}O—,

—[C(═O)]ᵥO(CH₂)ₜ(CH₂CH₂O)_{y}—,

—[C(═O)]ᵥO(CH₂)ₜ(CH₂CH₂O)_{y}NH—,

—[C(═O)]ᵥNH(CH₂CH₂O)ₜ(CH₂)_{y}O—,

—[C(═O)]ᵥNH(CH₂)ₜ(CH₂CH₂O)_{y}—,

—[C(═O)]ᵥNH(CH₂)ₜ(CH₂CH₂O)_{y}NH—,

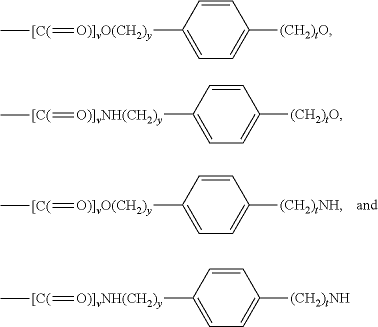

wherein (t), (t′) and (y) are independently chosen from zero or a positive integer, preferably from about 1 to about 10 (e.g., 1, 2, 3, 4, 5, and 6); and

(v) is 0 or 1.

In some aspects of the present invention, the compounds of Formula (I) (or Formula (II) or (III)) include from 1 to about 10 units (e.g., 1, 2, 3, 4, 5, or 6) of the bifunctional linker. In some preferred aspects of the present invention, the compounds include one unit of the bifunctional linker and thus (m) is 1.

Additional linkers are found in Table 1 of Greenwald et al. (Bioorganic & Medicinal Chemistry, 1998, 6:551-562), the contents of which are incorporated by reference herein.

3. Camptothecin and Related Camptothecin Analogs

Camptothecin is a water-insoluble cytotoxic alkaloid produced by camptoteca accuminata trees indigenous to China and nothapodytes foetida trees indigenous to India. Camptothecin and related compounds and analogs are also known to be potential anticancer or antitumor agents and have been shown to exhibit these activities in vitro and in vivo in laboratory animals. For example, camptothecin analogs useful in the treatment described herein includes SN38, camptothecin, topotecan, and CPT-11.

Camptothecin and certain related analogues share the structure:

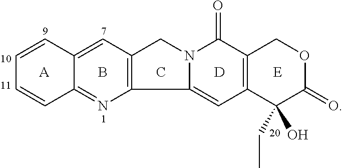

From this core structure, several known analogs have been prepared. For example, the A ring in either or both of the 10- and 11-positions can be substituted with an OH. The A ring can also be substituted with a straight or branched C₁₋₃₀ alkyl or C₁₋₁₇ alkoxy, optionally linked to the ring by a heteroatom i.e. —O or —S. The B ring can be substituted in the 7-position with a straight or branched C₁₋₃₀ alkyl (preferably C₂ alkyl), C₅₋₈ cycloakyl, C₁₋₃₀ alkoxy, phenyl alkyl, etc., alkyl carbamate, alkyl carbazides, phenyl hydrazine derivatives, etc. Other substitutions are possible in the C, D and E rings. See, for example, U.S. Pat. Nos. 5,004,758; 4,943,579; RE 32,518, the contents of which are incorporated herein by reference. As the artisan will appreciate, the 10-hydroxycamptothecin, 11-hydroxycamptothecin and the 10,11-dihydroxycamtothecin analogs occur naturally as one of the minor components in C. Acuminata and its relatives. Additional substitutions to these compounds, i.e. 7-alkyl-, 7-substituted alkyl-, 7-amino-, 7-aminoalkyl-, 7-aralkyl-, 9-alkyl-, 9-aralkyl- camptothecin etc. derviatives can be made using known synthetic techniques without undue experimentation.

Some camptotheca alkaloids have the structure shown below:

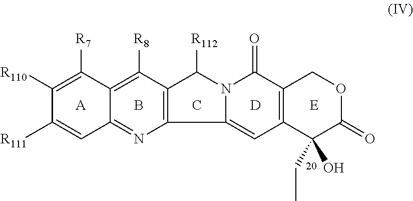

wherein

R₇ is selected among NO₂, NH₂, N₃, hydrogen, halogen, F, Cl, Br, I, COOH, OH, O—C₁₋₈ alkyl, SH, S—C₁₋₃ alkyl, CN, CH₂NH₂, NH—C₁₋₃ alkyl, CH₂—NH—C₁₋₃ alkyl, N(C₁₋₃ alkyl)₂, CH₂N(C₁₋₃ alkyl)₂, O—, NH— and S—CH₂CH₂N(CH₂CH₂OH)₂, O—, NH— and S—CH₂CH₂CH₂N(CH₂CH₂OH)₂, O—, NH— and S—CH₂CH₂N(CH₂CH₂CH₂OH)₂, O—, NH— and S—CH₂CH₂CH₂N(CH₂CH₂CH₂OH)₂, O—, NH— and S—CH₂CH₂N(C₁₋₃ alkyl)₂, O—, NH— and S—CH₂CH₂CH₂N(C₁₋₃ alkyl)₂, CHO and C₁₋₃ alkyl;

R₈ is selected among hydrogen, C₁₋₈ alkyl and CH₂NR₉R₁₀,
wherein
R₉ is selected from the group consisting of hydrogen, C₁₋₆ alkyl, C₃₋₇ cycloalkyl, C₃₋₇ cycloalkyl-C₁₋₆ alkyl, C₂₋₆ alkenyl, hydroxy-C₁₋₆ alkyl, and C₁₋₆ alkoxy-C₁₋₆ alkyl; and
R₁₀ is selected among hydrogen, C₁₋₆ alkyl, C₃₋₇ cycloalkyl, C₃₋₇ cycloalkyl-C₁₋₆ alkyl, C₂₋₆ alkenyl, hydroxy-C₁₋₆ alkyl, C₁₋₆ alkoxy-C₁₋₆ alkyl, and COR₁₁, wherein R₁₁ is selected from the group consisting of hydrogen, C₁₋₆ alkyl, perhalo-C₁₋₆ alkyl, C₃₋₇ cycloalkyl, C₃₋₇ cycloalkyl-C₁₋₆ alkyl, C₂₋₆ alkenyl, hydroxy-C₁₋₆ alkyl, C₁₋₆ alkoxy, and C₁₋₆ alkoxy-C₁₋₆ alkyl;

R₁₁₀-R₁₁₁ are each independently selected among hydrogen, halo, acyl, alkyl, substituted alkyl, alkoxy, substituted alkoxy, alkenyl, alkynyl, cycloalkyl, hydroxy, cyano, nitro, azido, amido, hydrazine, amino, substituted amino, hydroxcarbonyl, alkoxycarbonyl, alkylcarbonyloxy, alkylcarbonylamino, carbamoyloxy, arylsulfonyloxy, alkylsulfonyloxy, C(R₁₁₇)═N—(O)ⱼ-R₁₁₈ wherein R₁₁₇ is H, alkyl, alkenyl, cycloalkyl, or aryl, (j) is 0 or 1, and R₁₁₈ is H, alkyl, alkenyl, cycloalkyl, or heterocycloalkyl, and R₁₁₉C(O)O— wherein R₁₁₉ is halogen, amino, substituted amino, heterocycloalkyl, substituted heterocycloalkyl, or R₁₂₀-O—(CH₂)ₖ- where where (k) is an integer of 1-10 and R₁₂₀ is alkyl, phenyl, substituted phenyl, cycloalkyl, substituted cycloalkyl, heterocycloalkyl, or substituted heterocycloalkyl; or

R₇ together with R₁₁₀, or R₁₁₀ together with R₁₁₁, form substituted or unsubstituted methylenedioxy, ethylenedioxy, or ethyleneoxy; and

R₁₁₂ is H or OR′, wherein R′ is alkyl, alkenyl, cycloalkyl, haloalkyl, or hydroxyalkyl.

Preferred aryl groups are phenyl and naphthyl. Preferred heterocycloalkyl rings include bipiperidine. Suitable heterocyclic rings when R₉ and R₁₀ are taken together with the nitrogen atom to which they are attached include: aziridine, azetidine, pyrrolidine, piperidine, hexamethylenimine, imidazolidine, pyrazolidine, isoxazolidine, piperazine, N-methylpiperazine, tetrahydroazepine, N-methyl-tetrahydroazepine, thiazolidine, etc.

For ease of description and not limitation, the description refers to 7-ethyl-10-hydroxycamptothecin, or CPT-11 as the camptothecin analog, as the preferred and illustrated compound. It will be understood that the claimed invention includes all such derivatives and analogs so long as the analog has an OH, such as the 20-OH group, for the point of attachment to the polymer. The camptothecin or camptothecin analogs can be racemic mixtures or optically pure isomer. Preferably, a substantially pure and active form of such as the 20(S) camptothecin or camptothecin analog is employed in the multi-arm polymeric prodrugs.

4. Synthesis of Polymeric Compounds

Generally, the polymeric compounds employed in the treatment described herein are prepared by reacting one or more equivalents of an activated multi-arm polymer with, for example, one or more equivalents per active site of amino acid-(20)-7-ethyl-10-hydroxycamptothecin under conditions which are sufficient to effectively cause the amino group to undergo a reaction with the carboxylic acid of the polymer and form a linkage. Details of the synthesis are described in U.S. Pat. No. 7,462,627, the contents of which are incorporated herein by reference in its entirety.

Examples of preferred bifunctional linker groups include glycine, alanine, methionine, sarcosine, etc. and syntheses are described in the Examples of U.S. Pat. No. 7,462,627.

According to the present invention, the compounds administered include:

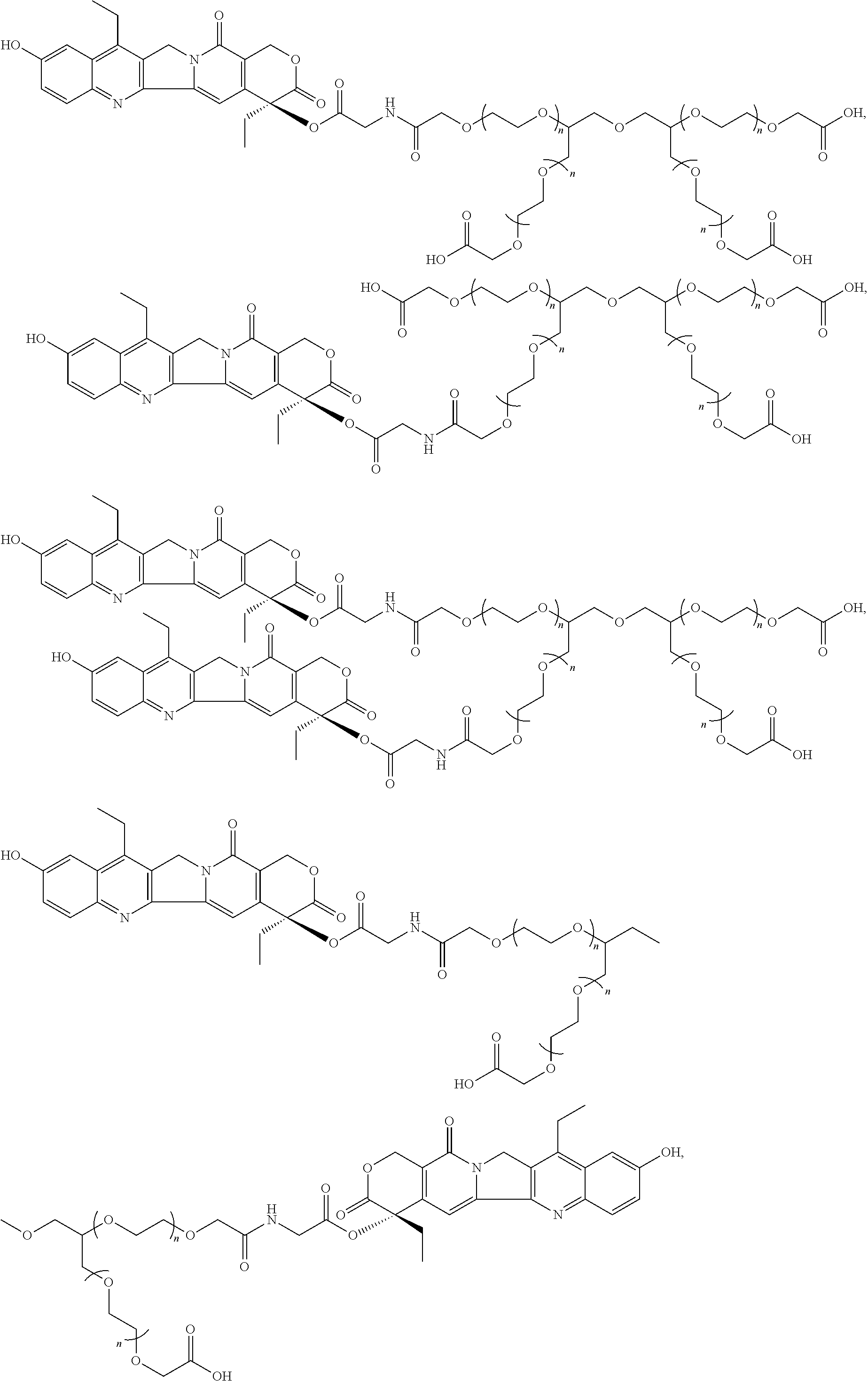

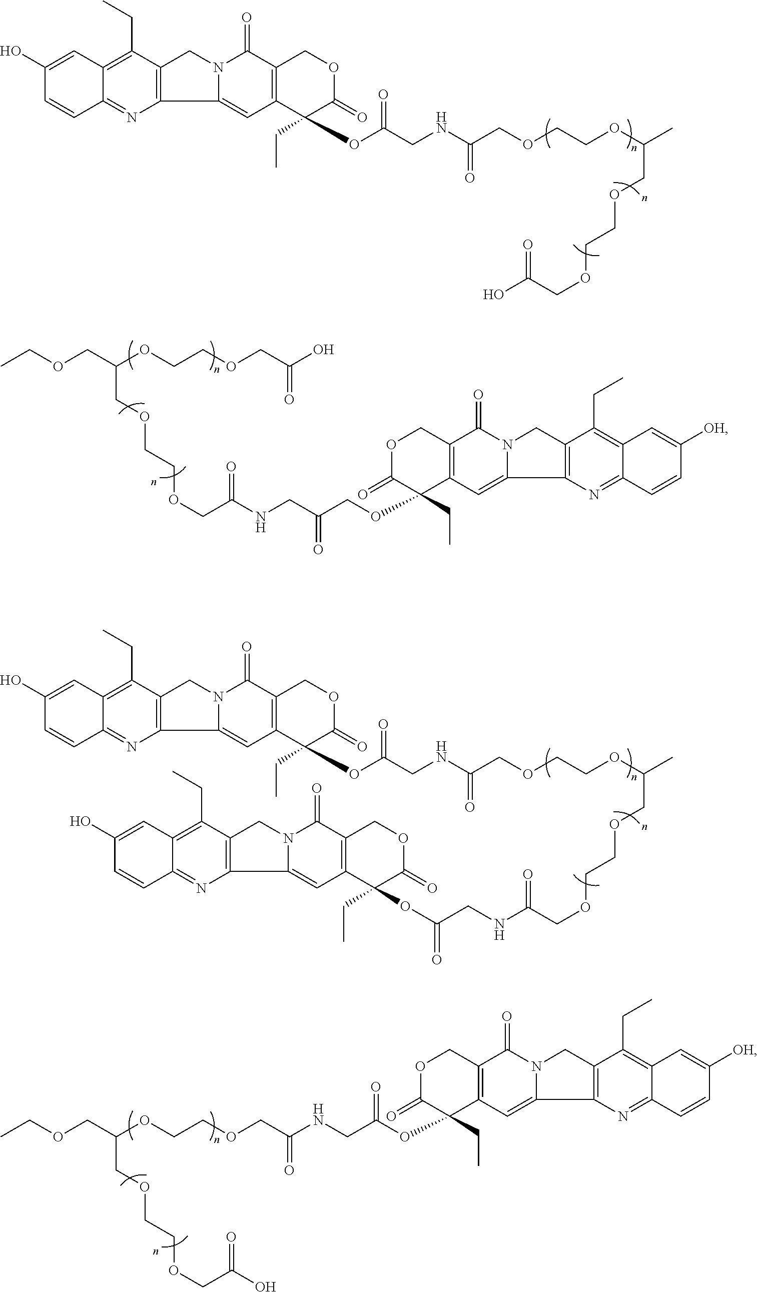

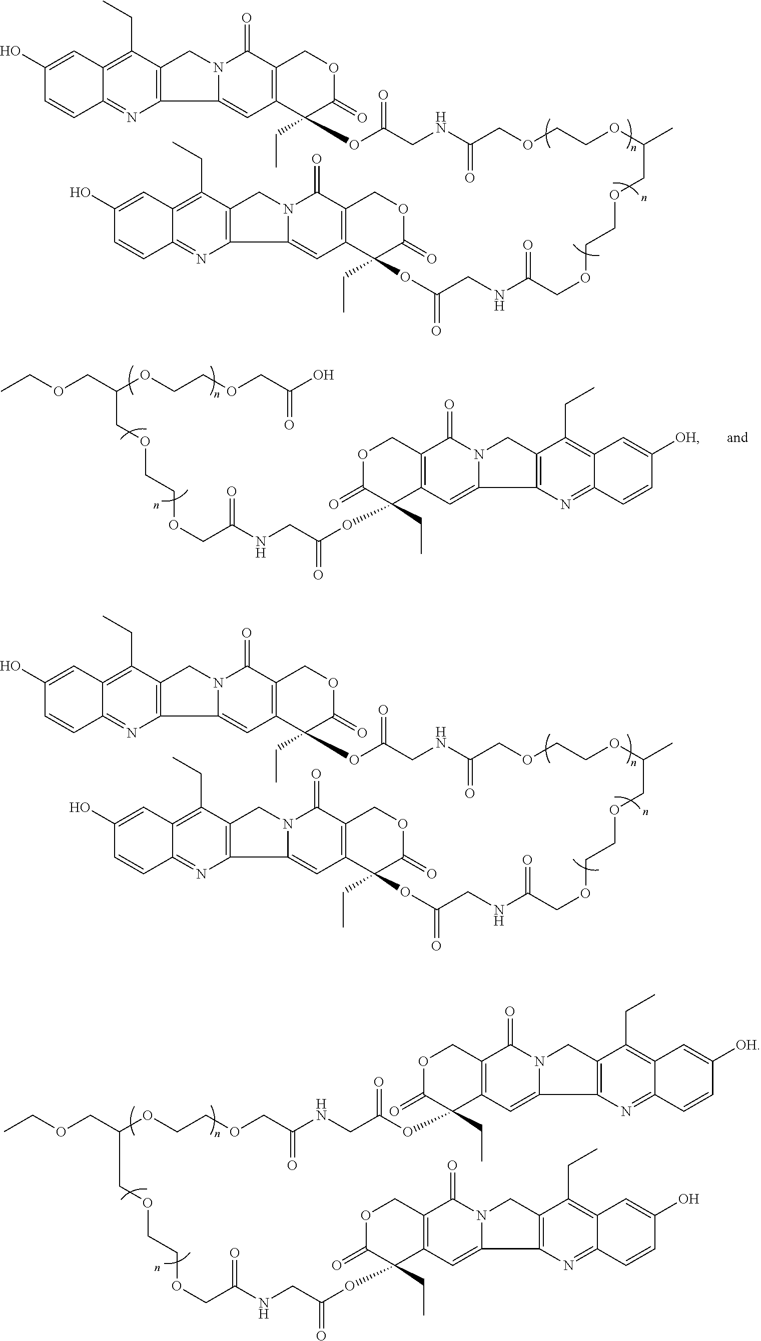

One particularly preferred embodiment includes administering a compound having the structure:

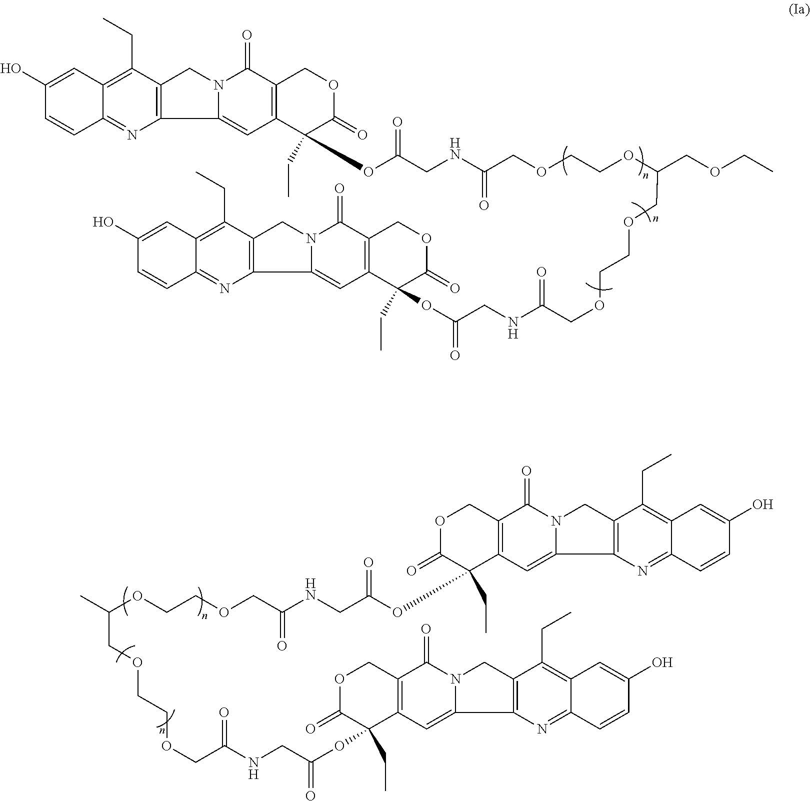

wherein all four arms of the polymer are conjugated to 7-ethyl-10-hydroxycamptothecin through glycine and the polymer portion has the total average molecular weight of about 40,000 daltons.

An alternative embodiment useful in the treatment described herein includes

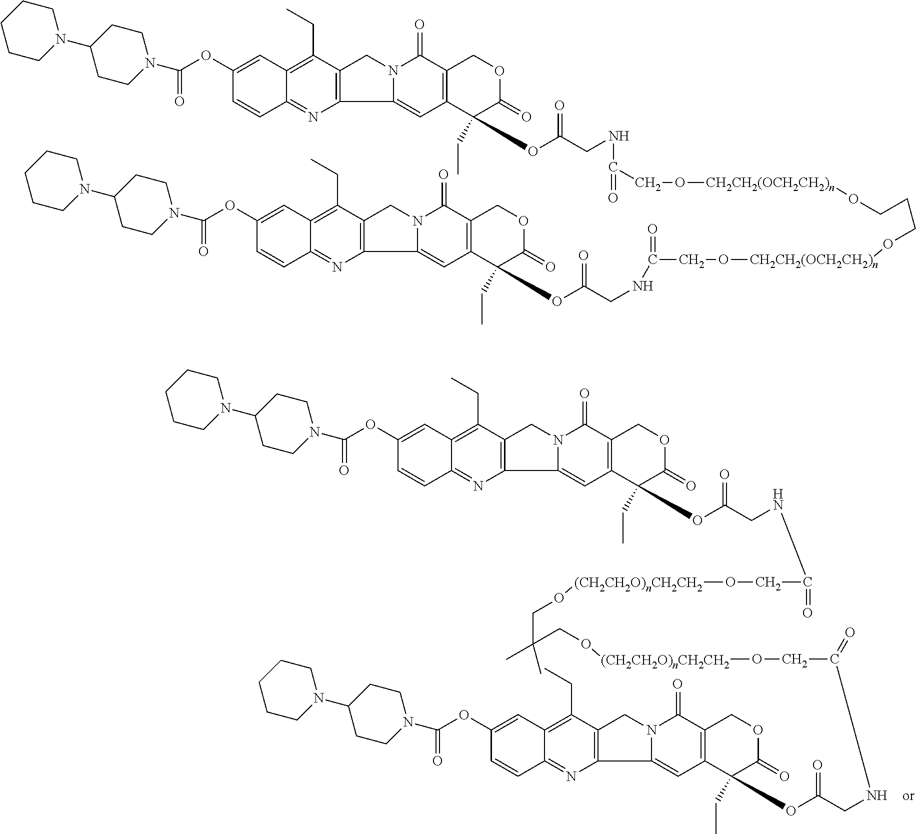

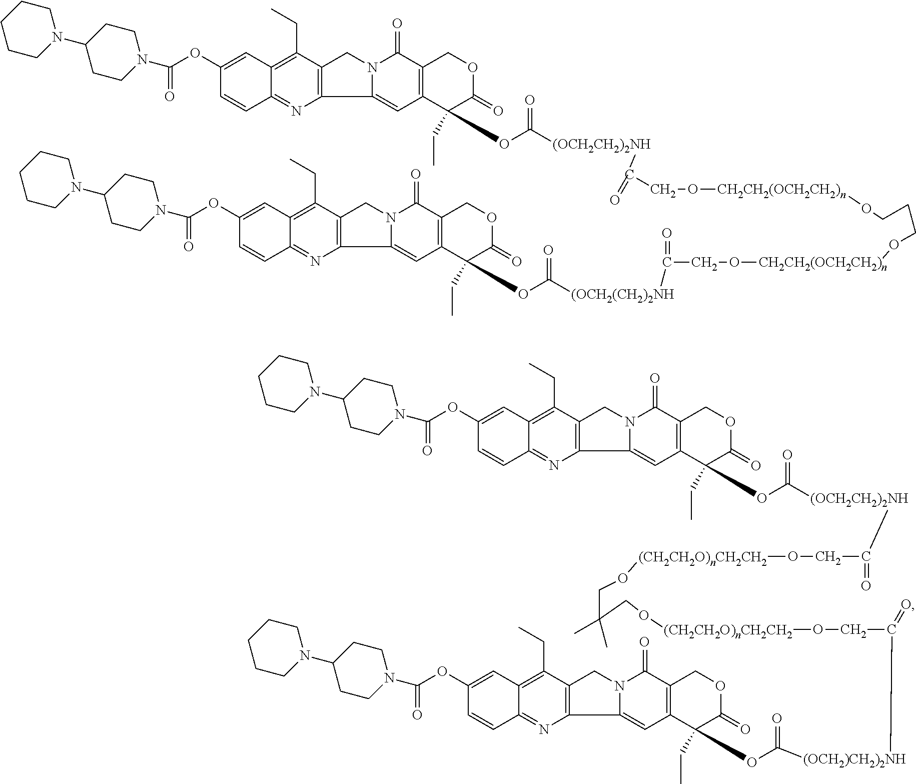

wherein (n) is an integer of from about 28 to about 341, so that the total molecular weight of the polymeric portion of the compound of Formula (II) ranges from about 5,000 to about 60,000 daltons, preferably about 20,000 or 40,000 daltons.

In a further embodiment, the treatment described herein employs polymeric compounds described in WO2005/028539, the contents of which are incorporated herein by reference in its entirety.

C. Her2 Antagonists

Many types of cancers have been associated with increased levels of the HER2 protein and gene. The HER2 protein catalyzes the transfer of the terminal phosphate from ATP to tyrosine residues of protein substrates. The HER2 receptor antagonist and HER2 antagonist generally refer to compounds which inhibit function or expression of the HER2 protein or gene. HER2 receptor antagonists can inhibit HER2 receptor function directly or via downstream or upstream cellular signaling pathway in which the HER2 protein is involved. In particular, HER2 receptor antagonists used in the combination treatment described herein includes anti-HER2 antibodies and antisense HER2 oligonucleotides which directly inhibit HER2 receptor function or expression of HER2 receptor, instead of inhibiting the HER2 receptor function via downstream or upstream signaling pathway.

In one embodiment, the combination treatment described herein is conducted by administering an anti-HER2 receptor antibody in combination with a compound of Formula (I) (or Formula (II) or (III)). The antibody binds to the HER2 receptor protein (p185). Preferably, the antibody useful in the treatment described herein binds to the extracellular domain of the HER2 receptor such as HER2 domain II and/or IV. One particular embodiment employs trastuzumab. Another particular embodiment employs pertuzumab.

Trastuzumab under the tradename Herceptin® is a recombinant humanized monoclonal antibody directed against the human epidermal growth factor receptor 2 (HER2). After binding to HER2 receptor on the tumor cell surface, trastuzumab induces an antibody-dependent cell-mediated cytotoxicity against tumor cells that overexpress HER2 receptor. HER2 is overexpressed by many adenocarcinomas, particularly breast adenocarcinomas. Trastuzumab is registered in CAS Registry No. 180288-69-1. Detailed information about trastuzumab is described in U.S. Pat. No. 6,165,464, the contents of which are incorporated herein by reference.

Pertuzumab under the tradename Omnitarg™ is also a recombinant humanized monoclonal antibody (2C4) directed against the extracellular dimerization domain of the HER2 receptor. (CAS Registry No. 380610-27-5). Pertuzumab binds to the dimerization domain of the HER2 receptor and inhibits the ability of the HER2 receptor protein to dimerize with other HER tyrosine kinase receptor proteins. The inhibition of receptor protein dimerization prevents the activation of HER signaling pathways, resulting in tumor cell apoptosis. Pertuzamab, also known as rhuMAb 2C4, is described, for example in U.S. Pat. Nos. 6,949,245 and 5,821,337, incorporated by reference herein.

In another embodiment, the methods described herein can be conducted wherein the compound of Formula (I) (or Formula (II) or (III)) is administered with antisense HER2 (ErbB2) oligonucleotides or pharmaceutically acceptable salt thereof. The antisense HER2 oligonucleotides can be administered concurrently or sequentially.

In one embodiment, the antisense HER2 oligonucleotide includes nucleic acids complementary to at least 8 consecutive nucleotides of HER2 pre-mRNA or mRNA.

An “oligonucleotide” is generally a relatively short polynucleotide, e.g., ranging in size from about 2 to about 200 nucleotides, or preferably from about 8 to about 50 nucleotides, or more preferably from about 8 to about 30 nucleotides. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties. The nucleic acids molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure.

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, incorporated by reference herein. The 3′-cap can include for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

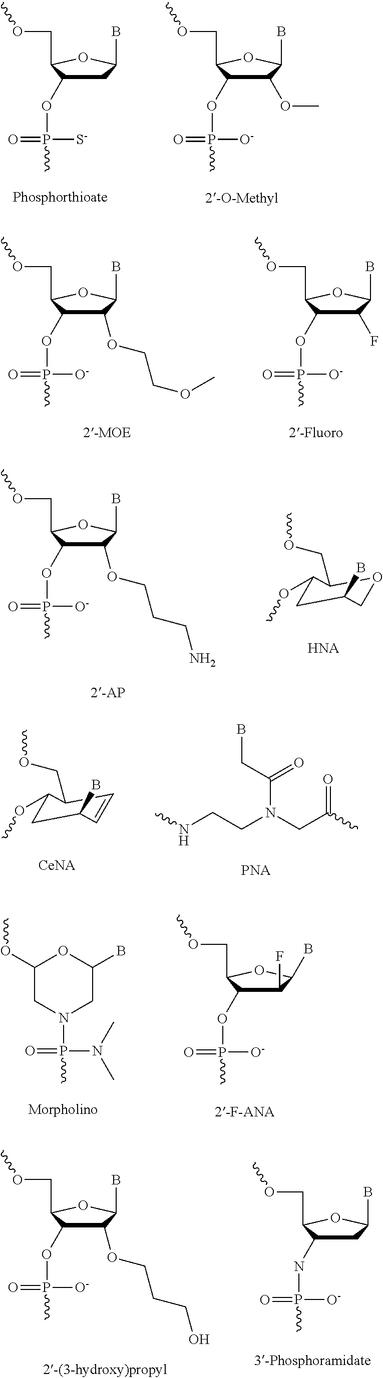

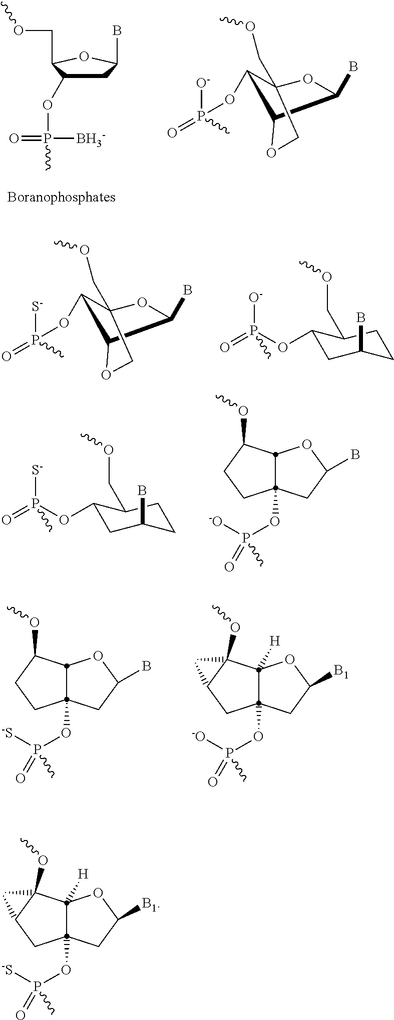

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The antisense strand serves as a template for synthesis of a messenger RNA (“mRNA”) transcript (a sense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same of contiguous residues in a second nucleic acid sequence.

The oligonucleotides or oligonucloetide derivatives useful in the method described herein can include from about 10 to about 1000 nucleic acids, and preferably relatively short polynucleotides, e.g., ranging in size from about 8 to about 30 nucleotides in length (e.g., about 8, 9, 10, 11, 12, 13, 14, 15, 16, 17, 18, 19, 20, 21, 22 23, 24, 25, 26, 27, 28, 29, or 30).

In one aspect of useful nucleic acids used in the method described herein, oligonucleotides and oligodeoxynucleotides with natural phosphorodiester backbone or phosphorothioate backbone or any other modified backbone analogues include:

LNA (Locked Nucleic Acid);

PNA (nucleic acid with peptide backbone);

short interfering RNA (siRNA);

microRNA (miRNA);

nucleic, acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

In another aspect of the nucleic acids used in the method described herein, oligonucleotides can optionally include any suitable art-known nucleotide analogs and derivatives, including those listed by Table 1, below:

TABLE 1
Representative Nucleotide Analogs And Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2-thiouridine |
| 5-methoxycarbonylmethyluridine | 5-carboxymethylaminomethyl-2- |
| | thiouridine |
| 5-methoxyuridine | 5-carboxymethylaminomethyluridine |
| Dihydrouridine | 2-methylthio-N6-isopentenyladenosine |
| 2′-O-methylpseudouridine | N-[(9-beta-D-ribofuranosyl-2- |
| | methylthiopurine-6-yl)carbamoyl] |
| | threonine |
| D-galactosylqueuosine | N-[(9-beta-D-ribofuranosylpurine-6-yl) |
| | N-methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine-6-yl)- |
| | carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

In one particular embodiment, the antisense HER2 (ErbB2) oligonucleotide includes nucleotides that are complementary to at least 8 consecutive nucleotides of the sequence set forth in SEQ ID NO: 1 (GenBank Accession No. X03363). See also, Yamamoto, T. et al. Nature 319:230-234, 1986; Papewalis, J. et al. Nucleic Acids Res. 1:5452, 1991, the contents of each of which are incorporated herein by reference in its entirety.

Preferably, the oligonucleotides according to the invention described herein include one or more phosphorothioate internucleotide linkages (backbone) and one or more locked nucleic acids (LNA). Preferably, LNA monomers include 2′-O, 4′-C methylene bicyclonucleotide as shown below:

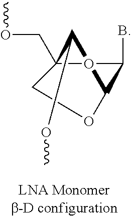

D. Selection of Patients with Her2 Positive Cancer

The treatment described herein benefits patients having a HER2 positive cancer. The treatment described herein significantly extends survival. Selection of patients having a HER2 positive cancer to receive the treatment described herein is predetermined by measuring levels of HER2 expression. In addition to HER2 expression levels, the patient's clinical history should be considered in selecting patients for the treatment described herein.

HER2 protein or gene levels can be measured by techniques known in the art, including, but not limited to, immunohistochemistry (IHC), silver in situ hybridization (SISH), chromogenic in situ hybridization (CISH), fluorescence in situ hybridization (FISH), virtual karyotyping, PCR-based methods, and other methods known in the art. Each type of assays has strength and limitations. Thus, selection of patients with a HER2 positive cancer can be more reliable when confirmed by a combination of the assays, rather than relying on a single assay to rule out potential benefit of the treatment described herein. Currently, the recommended assays are a combination of IHC and FISH.

Generally speaking, HER2 expression in cells or tissues can be assessed by measuring levels of HER2 receptor protein expression or HER2 gene amplification. FDA has approved several HER2 tests which aid to determine HER2 expression and there are several tests commercially available. These assays utilize IHC and/or FISH assays. For example, the commercially available assays include Dako HercepTest™ (Carpinteria, Calif., USA) and Ventana Pathway® HER-2/neu (AZ, USA), which measure the level of HER2 protein (IHC assays); and PathVysion® and HER2 FISH pharmDx™, which measure the level of gene amplication (FISH assays). Detailed information and guidelines for assessing HER2 expression in a test specimen are provided in the package insert of each of the test kits, the contents of the package insert of each of the aforementioned test kits are incorporated herein by reference. The assessment and validation of HER2 expression measurement should follow guidelines found in the package insert of the test kits.

Both HercepTest™ and Pathway™ test kits utilize IHC and measure levels of HER2 protein in a test tissue/cell. These are highly standardized, semi-quantitative assays. Interpretation of IHC test results use scoring system on a scale of 0 to 3+:0 (negative), 1+ (negative), 2+ (borderline/weak positive), or 3+ (positive), based on the reviewer's interpretation of staining intensity and completeness of cell membrane staining. Score 0 indicates that there are <20,000 receptors per cell, and that there is no visible membrane staining or membrane staining is observed in less than 10% of the tumor cells. Score 130 indicates there are ˜100,000 receptors per cell, that a faint membrane staining is observed in more than 10% of the tumor cells, but the membranes are partially stained. Score 230 indicates that there are ˜500,000 receptors per cell, that a weak to moderate complete membrane staining is observed in more than 10% of the tumor cells. Score 3+ indicates that there are ˜2,000,000 receptors per cell, and that a strong complete membrane staining is obtained in more than 10% of the tumor cells.

With FISH testing, tumors are interpreted as HER2 negative (FISH−) or positive (FISH+) by counting the HER2/neu gene copy . The presence of HER2 protein overexpression and gene amplification are highly correlated. FISH analysis reveals that some patients with IHC 2+ or IHC 3+ do not have gene amplification (FISH−), suggesting that these patients may be false positives. According to PathVysion® technology, approximately 40% of IHC-positive patients (2+/3+) did not have gene amplification, FISH negative. Thus, it is recommended that a patient with IHC 2+ be referred to a FISH test.

The combination treatment described herein is preferably given to IHC 2+ or 330 and/or FISH+ patients. Further, the combination treatment can be given to patients with a HER2 status comparable to IHC 2+ or 3+ and/or FISH+, when other techniques such as PCR methods are used for selection of HER2 positive patients.

E. Compositions/Formulations

Pharmaceutical compositions containing the polymer conjugates described herein and the HER2 antagonists may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention.

For injection, including, without limitation, intravenous, intramusclular and subcutaneous injection, the compounds of Formula (I) (alternatively, Formula (II) or (III)) described herein may be formulated in aqueous solutions, preferably in physiologically compatible buffers such as physiological saline buffer or polar solvents including, without limitation, a pyrrolidone or dimethylsulfoxide.

The compounds described herein may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use. In one embodiment, trastuzumab can be reconstituted with bacteriostatic water for injection.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl- methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

F. Dosages

For any compound used in the methods of the present invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the composition, e.g., used as a prodrug, that is administered will depend upon the parent molecule included therein (in this case, 7-ethyl-10-hydroxy-camptothecin). Generally, the amount of prodrug used in the methods described herein is that amount which effectively achieves the desired therapeutic result in mammals. Naturally, the dosages of the various prodrug compounds can vary somewhat depending upon the parent compound, rate of in vivo hydrolysis, molecular weight of the polymer, etc. In addition, the dosage, of course, can vary depending upon the dosage form and route of administration.

In general, however, the polymeric ester derivatives of 7-ethyl-10-hydroxy-camptothecin described herein can be administered in amounts ranging from about 0.3 to about 90 mg/m² body surface, and preferably from about 0.5 to about 50 mg/ m² body surface/dose, yet preferably from about 1 to about 18 mg/ m² body surface/dose, and even more preferably from about 1.25 mg/m² body surface/dose to about 16.5 mg/m² body surface/dose for systemic delivery. Some particular doses include one of the following: 1.25, 2.5, 5, 9, 10, 12, 13, 14, 15, 16 and 16.5 mg/m²/dose. One preferred dosage includes 5 mg/m² body surface/dose.

The compounds of Formula (I) (or Formula (II) or (III)) described herein can be administered in amounts ranging from about 0.3 to about 90 mg/ m² body surface/week such as, for example, from about 1 to about 18 mg/ m² body surface/week. In particular embodiments, the dose regimens can be, for example, from about 5 to about 7 mg/m² body surface weekly for 3 weeks in 4-week cycles, from about 1.25 to about 45 mg/m² one injection every 3 weeks, and/or from about 1 to about 16 mg/m² three injections weekly in a four week cycle.

The treatment protocol can be based, for example, on a single dose administered once every three weeks or divided into multiple doses which are given as part of a multi-week treatment protocol. Thus, the treatment regimens can include, e.g., one dose every three weeks for each treatment cycle and, alternatively one dose weekly for three weeks followed by one week off for each cycle. It is also contemplated that the treatment will be given for one or more cycles until the desired clinical result is obtained.

The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. The precise dose will depend on the stage and severity of the condition, and the individual characteristics of the patient being treated, as will be appreciated by one of ordinary skill in the art.

Additionally, toxicity and therapeutic efficacy of the compounds described herein can be determined by standard pharmaceutical procedures in cell cultures or experimental animals using methods well-known in the art.

In some preferred embodiments, the treatment protocol includes administering the amount ranging from about 1.25 to about 16.5 mg/m² body surface/dose weekly for three weeks, followed by one week without treatment and repeating for about 3 cycles or more until the desired results are observed. The amount administered per each cycle can range from about 2.5 to about 16.5 mg/m² body surface/dose.

In one particular embodiment, the polymeric ester derivatives of 7-ethyl-10-hydroxycamptothecin can be administered in one dose, such as 5, 9 or 10 mg/m² weekly for three weeks, followed by one week without treatment. The dosage of the treatment cycle can be designed as an escalating dose regimen when two or more treatment cycles are applied. The polymeric drug is preferably administered via IV infusion.

In another particular embodiment, the compound of Formula (I) (or Formula (II) or (III)) is administered in a dose from about 12 to about 16 mg/m² body surface/dose. The dose can be given weekly. The treatment protocol includes administering the compound of Formula (I) (or Formula (II) or (III)) in amounts ranging from about 12 to about 16 mg/m² body surface/dose weekly for three weeks, followed by one week without treatment.

In yet another particular embodiment, the dose regiment can be about 10 mg/m² body surface/dose every three weeks.

Alternative embodiments include: for the treatment of pediatric patients, a regimen based on a protocol of about 1.85 mg/m² body surface/dose daily for 5 days every three weeks, a protocol of from about 1.85 to about 7.5 mg/m² body surface/dose daily for 3 days every 25 days, or a protocol of about 22.5 mg/m² body surface/dose once every three weeks, and for the treatment of adult patients, a protocol based on about 13 mg/m² body surface/dose every three weeks or about 4.5 mg/m² body surface/dose weekly for four weeks every six weeks. The compounds described herein can be administered in combination with a second therapeutic agent. In one embodiment, the combination therapy includes a protocol of about 0.75 mg/m² body surface/dose daily for 5 days each cycle in combination with a second agent.

Alternatively, the compounds can be administered based on body weight. The dosage range for systemic delivery of a compound of Formula (I) (or Formula (II) or (III)) in a mammal will be from about 1 to about 100 mg/kg/week and is preferably from about 2 to about 60 mg/kg/week. Thus, the amounts can range from about 0.1 mg/kg body weight/dose to about 30 mg/kg body weight/dose, preferably, from about 0.3 mg/kg to about 10 mg/kg. Specific doses such as 5 or 10 mg/kg at q2d×5 regimen (multiple dose) or 20 or 30 mg/kg on a single dose regimen can be administered.

In all aspects of the invention where polymeric conjugates are administered, the dosage amount mentioned is based on the amount of an active agent (preferably, 7-ethyl-10-hydroxycamptothecin) rather than the amount of polymeric conjugate administered. It is contemplated that the treatment will be given for one or more cycles until the desired clinical result is obtained. The exact amount, frequency and period of administration of the compound of the present invention will vary, of course, depending upon the sex, age and medical condition of the patient as well as the severity of the disease as determined by the attending clinician. The weight given above represents the weight of 7-ethyl-10-hydroxycamptothecin present in the PEG-conjugated 7-ethyl-10-hydroxy-camptothecin employed for treatment. The actual weight of the PEG-conjugated 7-ethyl-O-hydroxycamptothecin will vary depending on the loading of the PEG (e.g., optionally from one to four moles of 7-ethyl-10-hydroxycamptothecin per mole of PEG.).

The HER2 antagonists can be administered in combination with the compound of Formula (I) (or Formula (II) or (III)) concurrently or sequentially. The combination therapy protocol includes administering an anti-HER2 antibody ranging from about 0.5/kg to about 15 mg/kg body weight, i.e., from about 2 mg/kg to about 8 mg/kg/dose such as 2, 4, 5, 6, 8 mg/kg/dose.

In one embodiment, trastuzumab is administered based on a protocol: initial dose at 4 mg/kg i.v. followed by 2 mg/kg/dose i.v. weekly during and after the combination therapy, or initial dose at 8 mg/kg i.v. followed by 6 mg/kg i.v. every three weeks, until a desired clinical result is achieved. Detailed dosing information of trastuzumab is described in the package insert of Herceptin®, the contents of which are incorporated herein by reference.

In another embodiment, pertuzumab is administered in an amount raging from about 0.5 to about 15 mg/kg/dose i.v. every three weeks during and after the combination treatment described herein. Pertuzumab is given based on a protocol: 5 mg/kg/dose every three weeks. See Agus, D. B., et al., Journal of Clinical Oncology, 23:2534-2543, 2005, the contents of which are incorporated herein by reference.

The combination therapy protocol includes administering an antisense oligonucleotide in an amount of from about 2 to about 100 mg/kg/dose (e.g., 2, 3, 4, 5, 6, 8, 10, 15, 20, 25, 30, 35, 40, 50, 60, 70, 100 mg/kg/dose). For example, the combination therapy regimen dose includes treatment with an antisense HER2 oligonucleotide in an amount of from about 2 to about 50 mg/kg/dose. Preferably, the antisense oligonucleotide administered in the combination therapy is in an amount of from about 4 to about 25 mg/kg/dose.

In one aspect of the combination therapy, the protocol includes administering an antisense HER2 oligonucleotide in an amount of about 4 to about 18 mg/kg/dose weekly, or about 4 to about 9.5 mg/kg/dose weekly during the combination therapy.

In one particular embodiment, the combination therapy protocol includes an antisense HER2 oligonucleotide in an amount of about 4 to about 18 mg/kg/dose weekly for 3 weeks in a six week cycle (i.e. about 8 mg/kg/dose). Another particular embodiment includes about 4 to about 9.5 mg/kg/dose weekly (i.e., about 4.1 mg/kg/dose). Where the HER2 antagonists encompassed by the present invention are administered in combination with the compounds of Formula (I) (or Formula (II) or (III)) described herein, the individual components of the combinations may be administered either sequentially or simultaneously in separate or combined pharmaceutical formulations by any convenient route. When the HER2 antagonist and the compound of Formula (I) (or Formula (II) or (III)) is administered sequentially, either the compound of Formula (I) (or Formula (II) or (III)) or the HER antagonist may be administered first. For example, the HER2 antagonist and the compound of Formula (I) (or Formula (II) or (III)) may be administered in a sequential manner in a regimen that will provide beneficial effects of the combination. When the HER2 antagonist and the compound of Formula (I) (or Formula (II) or (III)) is administered in a simultaneous manner, the combination may be administered either in the same or different pharmaceutical compositions.

Further aspects of the present invention include combining the HER2 antagonist and the compounds described herein with other chemotherapy or radiotherapy for additive benefit.

### EXAMPLES

The following examples serve to provide further appreciation of the invention but are not meant in any way to restrict the effective scope of the invention.

### Example 1

Toxicity Data

A maximum tolerated dose (“MTD”) of 4arm-PEG-Gly-(7-ethyl-10-hydroxycamptothecin) (compound 9) was studied using nude mice. Mice were monitored for 14 days for mortality and signs of illness and sacrificed when body weight loss was >20% of the pretreatment body weight.

Table 2, below, shows the maximum tolerated dose of each compound for both single dose and multiple dose administration. Each dose for multiple dose administration was given mice every other day for 10 days and the mice were observed for another 4 days, thus for total 14 days.

TABLE 2
MTD Data in Nude Mice

| | Dose Level | Survival/ | |
|---|---|---|---|
| Compound | (mg/kg) | Total | Comments |
| Compound 9 | 25 | 5/5 | |
| Single dose | 30 | 5/5 | |
| | 35 | 4/5 | Mouse euthanized due to >20% |
| | | | body weight loss |
| Compound 9 | 10 | 5/5 | |
| Multiple dose* | 15 | 3/5 | Mice euthanized due to >20% |
| | | | body weight loss |
| | 20 | 0/5 | Mice euthanized due to >20% |
| | | | body weight loss |

The MTD found for 4arm-PEG-Gly-(7-ethyl-10-hydroxycamptothecin) (compound 9) was 30 mg/kg when given as single dose, and 10 mg/kg when given as multiple dose (q2d×5).

### Example 2

Properties of PEG Conjugates

Table 3, below, shows solubility of four different PEG-(7-ethyl-10-hydroxycamptothecin) conjugates in aqueous saline solution. All four PEG-(7-ethyl-10-hydroxycamptothecin) conjugates showed good solubility of up to 4 mg/mL equivalent of 7-ethyl-10-hydroxycamptothecin. In human plasma, 7-ethyl-10-hydroxycamptothecin was steadily released from the PEG conjugates with a doubling time of 22 to 52 minutes and the release appeared to be pH and concentration dependent as described in the following

### Example 3

TABLE 3
Properties of PEG-7-ethyl-10-hydroxycamptothecin Conjugates

| | Solubility | t _{1/2}(min) in | Doubling Time | | |
|---|---|---|---|---|---|
| | in Saline | Human | in Plasma (min)^{c} | | |
| Compound | (mg/mL)^{a} | Plasma^{b} | Human | Mouse | Rat |
| Compound 9 | 180 | 12.3 | 31.4 | 49.5 | 570 |
| (Gly) | | | | | |
| Compound 12 | 121 | 12.5 | 51.9 | 45.8 | 753 |
| (Ala) | | | | | |
| Compound 23 | ND | 19.0 | 28.8 | 43.4 | 481 |
| (Sar) | | | | | |
| Compound 18 | 142 | 26.8 | 22.2 | 41.9 | 1920 |
| (Met) | | | | | |

^{a}7-ethyl-10-hydroxycamptothecin is not soluble in saline.
^{b}PEG conjugate half life.
^{c}7-ethyl-10-hydroxycamptothecin formation rate from conjugates.

PEG-7-ethyl-10-hydroxycamptothecin conjugates show good stability in saline and other aqueous medium for up to 24 hours at room temperature.

### Example 4

Effects Of Concentration and pH on Stability

Based on our previous work, acylation at the 20-OH position protects the lactone ring in the active closed form. The aqueous stability and hydrolysis properties in rat and human plasma were monitored using UV based HPLC methods. 4armPEG-Gly-(7-ethyl-10-hydroxycamptothecin) conjugates were incubated with each sample for 5 minutes at room temperature.

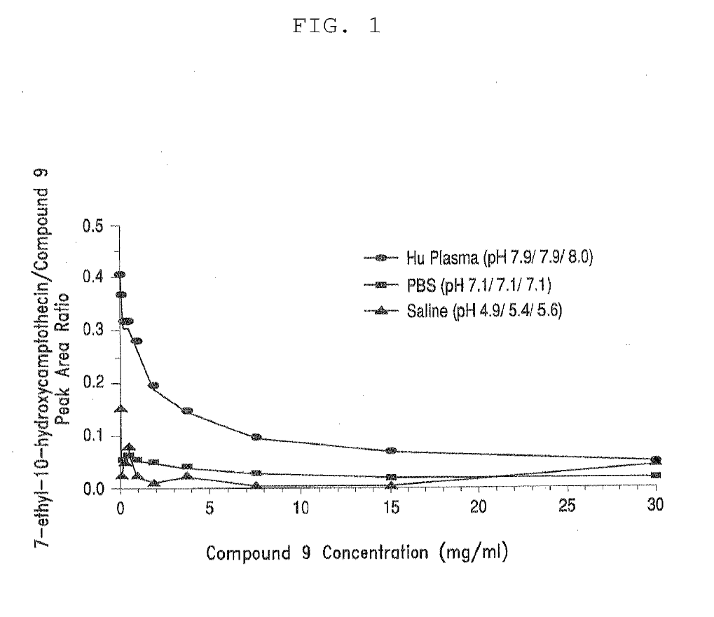

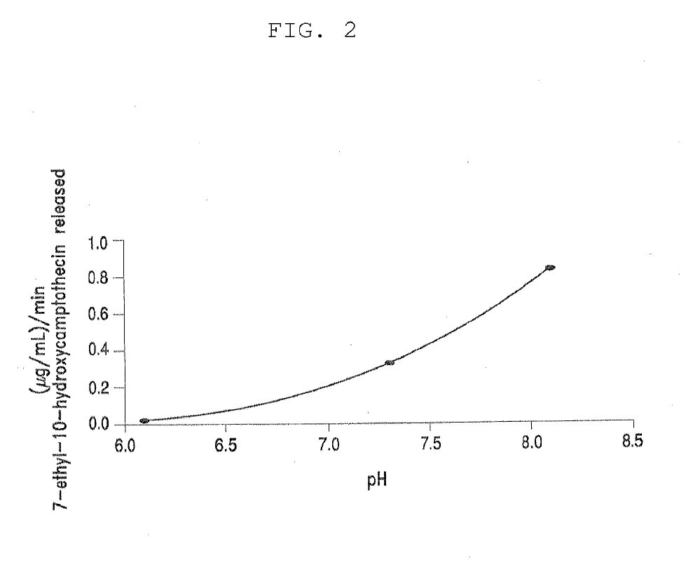

Stability of PEG-7-ethyl-10-hydroxycamptothecin conjugates in buffer was pH dependent. FIG. 1 shows 4armPEG-Gly-(7-ethyl-10-hydroxycamptothecin) stability in various samples. FIG. 2 shows that the rate of 7-ethyl-10-hydroxycamptothecin release from PEG-Gly-(7-ethyl-10-hydroxycamptothecin) increases with increased pH.

### Example 5

Pharmacokinetic Properties

Tumor free Balb/C mice were injected with a single injection of 20 mg/kg 4armPEG-Gly-(7-ethyl-10-hydroxycamptothecin) conjugates. At various time points mice were sacrificed and plasma was analyzed for intact conjugates and released 7-ethyl-10-hydroxycamptothecin by HPLC. Pharmacokinetic analysis was done using non-compartmental analysis (WinNonlin). Details are set forth in Table 4, below.

TABLE 4
Pharmacokinetic Data

| | | 7-ethyl-10-hydroxy- |
|---|---|---|
| | | camptothecin Released |
| Parameter | Compound 9 | from Compound 9 |
| AUC (h * μg/mL) | 124,000 | 98.3 |
| Terminal t _{1/2} (Hr) | 19.3 | 14.2 |
| Cₘₐₓ (μg/mL) | 20,500 | 13.2 |
| CL(mL/hr/kg) | 5.3 | 202 |
| Vss (mL/kg) | 131 | 3094 |

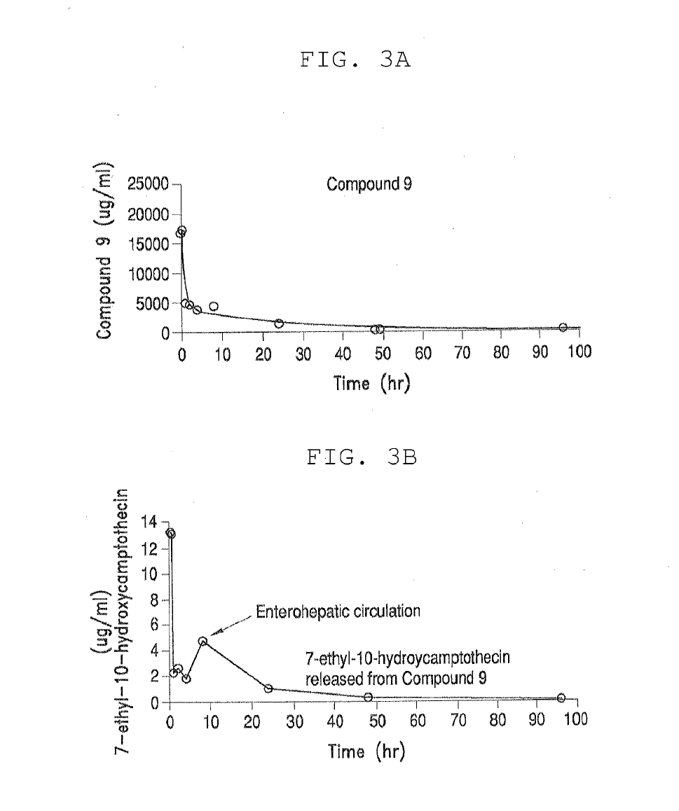

As shown in FIGS. 3A and 3B, PEGylation of 7-ethyl-10-hydroxycamptothecin allows long circulation half life and high exposure to native drug 7-ethyl-10-hydroxycamptothecin. Enterohepatic circulation of 4armPEG-Gly-(7-ethyl-10-hydroxycamptothecin) conjugates was observed. The pharmacokinetic profile of PEG-Gly-(7-ethyl-10-hydroxycamptothecin) in mice was biphasic showing a rapid plasma distribution phase during the initial 2 hours followed by a 18-22 hours terminal elimination half-life for the conjugate and a concomitant 18-26 hours terminal elimination half-life for 7-ethyl-10-hydroxycamptothecin (FIG. 3B).

Additionally, pharmacokinetic profiles of 4arm PEG-Gly-(7-ethyl-10-hydroxycamptothecin) were investigated in rats. In rats, dose levels of 3, 10 and 30 mg/kg (7-ethyl-10-hydroxycamptothecin equivalent) were used. The pharmacokinetic profiles in rats were consistent with those of mice.

In rats, 4 arm PEG-Gly-(7-ethyl-10-hydroxycamptothecin) showed a biphasic clearance from the circulation with an elimination half life of 12-18 hours in rats. 7-ethyl-10-hydroxycamptothecin released from 4armPEG-Gly-7-ethyl-10-hydroxycamptothecin conjugates had an apparent elimination half life of 21-22 hours. The maximum plasma concentration (Cₘₐₓ) and area under the curve (AUC) increased in a dose dependent manner in rats. The apparent half life of released 7-ethyl-10-hydroxycamptothecin from 4 armPEG-Gly conjugates in mice or rats is significantly longer than the reported apparent half life of released 7-ethyl-10-hydroxycamptothecin from CPT-11 and the exposure of released 7-ethyl-10-hydroxycamptothecin from 4 arm PEG-Gly-(7-ethyl-10-hydroxycamptothecin) is significantly higher than the reported exposure of released 7-ethyl-10-hydroxycamptothecin from CPT-11. The clearance of the parent compound was 0.35 mL/hr/kg in rats. The estimated volume of distribution at steady state (Vss) of the parent compound was 5.49 mL/kg. The clearance of the released 7-ethyl-10-hydroxycamptothecin was 131 mL/hr/kg in rats. The estimated Vss of released 7-ethyl-10-hydroxycamptothecin was 2384 mL/kg in rats. Enterohepatic circulation of released 7-ethyl-10-hydroxycamptothecin was observed both in mice and rats.

### Example 6

Therapeutic Efficacy in Human Breast Tumor Xenografted Mice Refractory to Herceptin®

Therapeutic efficacy of HER2 receptor antagonist-containing therapies against a refractory human JIMT-1 breast tumor grown in nude mice was measured. Tumors were established by implanting small tumor fragments into a single subcutaneous site on the left auxiliary flank region of nude mice. The tumor implantation site was observed twice weekly and measured once palpable. The tumor volume for each mouse was determined by measuring two dimensions with calipers and calculated. When tumors reached an average volume of 100 mm³, the mice were divided into their experimental groups consisting of: untreated controls, Herceptin® only, compound 9 only, and a combination of compound 9 and Herceptin®. Herceptin 12 was given 5 mg/kg body weight/dose at q7d intraperitoneally. Compound 9 was given 4 mg/kg/dose at q2d×5 intravenously. For the combination treatment, Herceptin® and compound 9 were given 5 mg/kg/dose at q7d and 4 mg/kg/dose at q2d×5, respectively. Compound 9 was administered 1 minute following Herceptin® treatment on day 0. On no other day did treatment of compound 9 and Herceptin® coincide. In these experiments, the amount of compound 9 administered was based on the amount of 7-ethyl-10-hydroxycamptothecin, not the amount of polymeric conjugate administered. Mouse weight and tumor sizes were measured at the beginning of the study and at day 28.

The treatment with Herceptin® and compound 9 alone led to about 28% and 51% TGI, respectively as of day 28. The treatment with the combination of Herceptin® and compound 9 resulted in about 81% TGI. The results are set forth in Table 5.

TABLE 5
Therapeutic Efficacy in JIMT-1 Breast
Tumor Xenografted Mouse Model

| Treatment | TGI (%) |
|---|---|
| Control | — |
| Herceptin ® i.p. | 28.34 |
| compound 9 i.v. | 50.74 |
| Herceptin ® i.p. & compound 9 i.v. | 80.80 |

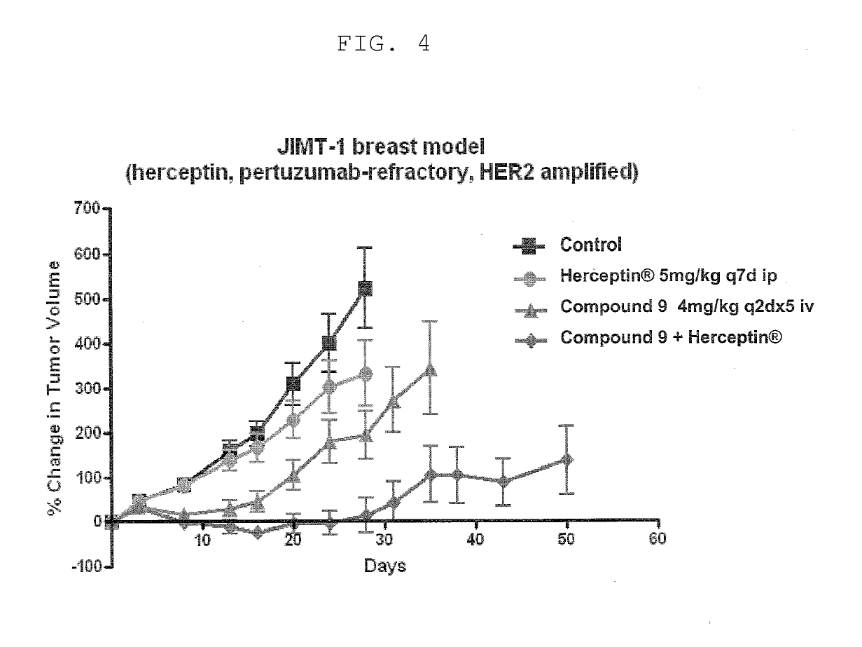

Tumor volume measured at various time points are shown in FIG. 4. A combination of compound 9 and Herceptin® inhibited tumor growth significantly as compared to that of compound 9 or Herceptin ⁱⁱ alone. The results show that Herceptin®, when administered with compound 9, is significantly more effective than either Herceptin® or compound 9 alone in the treatment of breast cancer.

The therapy using compounds described herein in combination with the HER2 receptor antagonist unexpectedly ameliorates and/or avoids resistance associated with HER2 antagonist-containing therapy. The human JIMT-1 breast tumor is refractory to a HER2 antibody such as trastuzumab and pertuzumab. The therapy described herein provides ways to treat cancers refractory to HER2 antagonists more effectively by avoiding and reducing potential drug resistance. Patients and clinicians can benefit from unexpected lack and/or reduction of resistance to HER2 antagonist-containing therapy, when a HER2 antagonist is administered together with the compounds described herein.

### Example 7

Therapeutic Efficacy in Human Gastric Carcinoma Xenografted Mice

Therapeutic efficacy of HER2 receptor antagonist-containing therapies against a human gastric carcinoma N87 grown in nude mice was determined. Human N87 gastric carcinoma was established in nude mice by subcutaneous injection. Groups of mice'were randomly divided and treated with Herceptin® alone, compound 9 alone, and a combination of both. Herceptin 1z was given 20 mg/kg body weight/dose at q7d intraperitoneally. Compound 9 was given 5 mg/kg/dose at q2d×5 intravenously. For the combination treatment, compound 9 and Herceptin® were given 5 mg/kg/dose at q2d×5 and 20 mg/kg/dose at q7d, respectively. The amounts of compound 9 administered were based on the amount of 7-ethyl-10-hydroxycamptothecin.

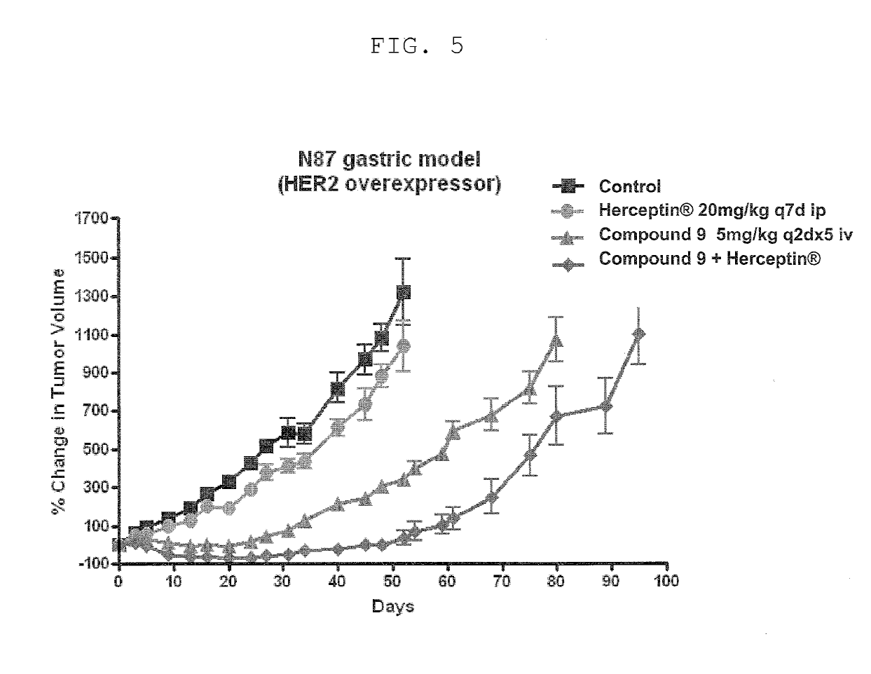

The results are set forth in FIG. 5. Tumors continued to grow in the mice treated with Herceptin® alone. On day 40, tumor volume increased by 613% compared to day 0. The tumor volume in the mice treated with Herceptin® alone was comparable to the control untreated mice. Herceptin® alone did not inhibit gastric tumor growth. The treatment with compound 9 alone inhibited tumor growth effectively. In the mice treated with the combination of Herceptin® and compound 9, tumor volume decreased by 23% by day 40 compared to day 0. The tumors receiving combined treatment regressed (below baseline values) from day 5 until day 48 of the study. 71% of animals treated with Herceptin® alone were sacrificed by day 52 due to excessive tumor burden (>1700 mm³) or tumor ulceration. In the group treated with Herceptin® plus compound 9, 86% of mice survived until day 95 (the last day of the study). Among the 5 surviving mice (71%) treated with four-arm ^{40K}PEG-Gly-(7-ethyl-10-hydroxycamptothecin), all had tumors <1500 mm³ by day 80.

The results show that therapeutic efficacy of the HER2 receptor antagonist and survival rate, when administered in combination with the compounds described herein, were enhanced significantly. The treatment described herein provides ways to utilize HER2 antagonist-based therapy more effectively.

Various references are cited herein. The contents of all of which are hereby incorporated by reference herein in their entireties.

We claim:

1. A method of treating a HER2 positive cancer in a mammal, comprising administering a HER2 receptor antagonist in combination with an effective amount of a compound of Formula (I): 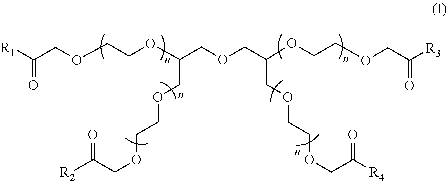
wherein
R₁, R₂, R₃ and R₄ are independently OH or 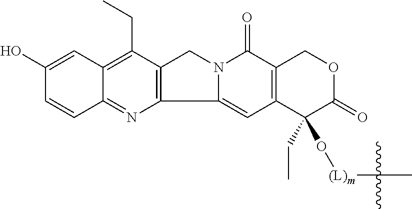
wherein
L is a bifunctional linker;
(m) is 0 or a positive integer, wherein each L is the same or different when (m) is equal to or greater than 2; and
(n) is a positive integer;
provided that R₁, R₂, R₃ and R₄ are not all OH;
or a pharmaceutically acceptable salt thereof, to said mammal.

2. The method of claim 1, wherein the HER2 receptor antagonist is selected from the group consisting of anti-HER2 antibodies, antisense ErbB2 oligonucleotides, and combinations thereof.

3. The method of claim 2, wherein the anti-HER2 antibodies bind to the extracellular domain of HER2 Domain IV or HER2 Domain II.

4. The method of claim 2, wherein the anti-HER2 antibody comprises trastuzumab or pertuzumab.

5. The method of claim 1, wherein the HER2 positive cancer is metastatic or non-metastatic.

6. The method of claim 1, wherein the HER2 positive cancer is resistant or refractory to the HER2 receptor antagonist.

7. The method of claim 1, wherein the HER2 positive cancer is selected from the group consisting of solid tumors, breast cancer, gastric cancer, ovarian cancer, stomach cancer, uterine cancer, uterine serous endometrial carcinoma, prostate cancer, bladder cancer, salivary gland carcinoma, renal adenocarcinoma, and mammary gland carcinoma.

8. The method of claim 1, wherein (n) is an integer of from about 28 to about 341, so that the total molecular weight of the polymeric portion of the compound of Formula (I) ranges from about 5,000 to about 60,000 daltons.

9. The method of claim 8, wherein (n) is an integer of from about 114 to about 239, so that the total molecular weight of the polymeric portion of the compound of Formula (I) ranges from about 20,000 to about 42,000 daltons.

10. The method of claim 1, wherein the compound of Formula (I) is selected from the group consisting of

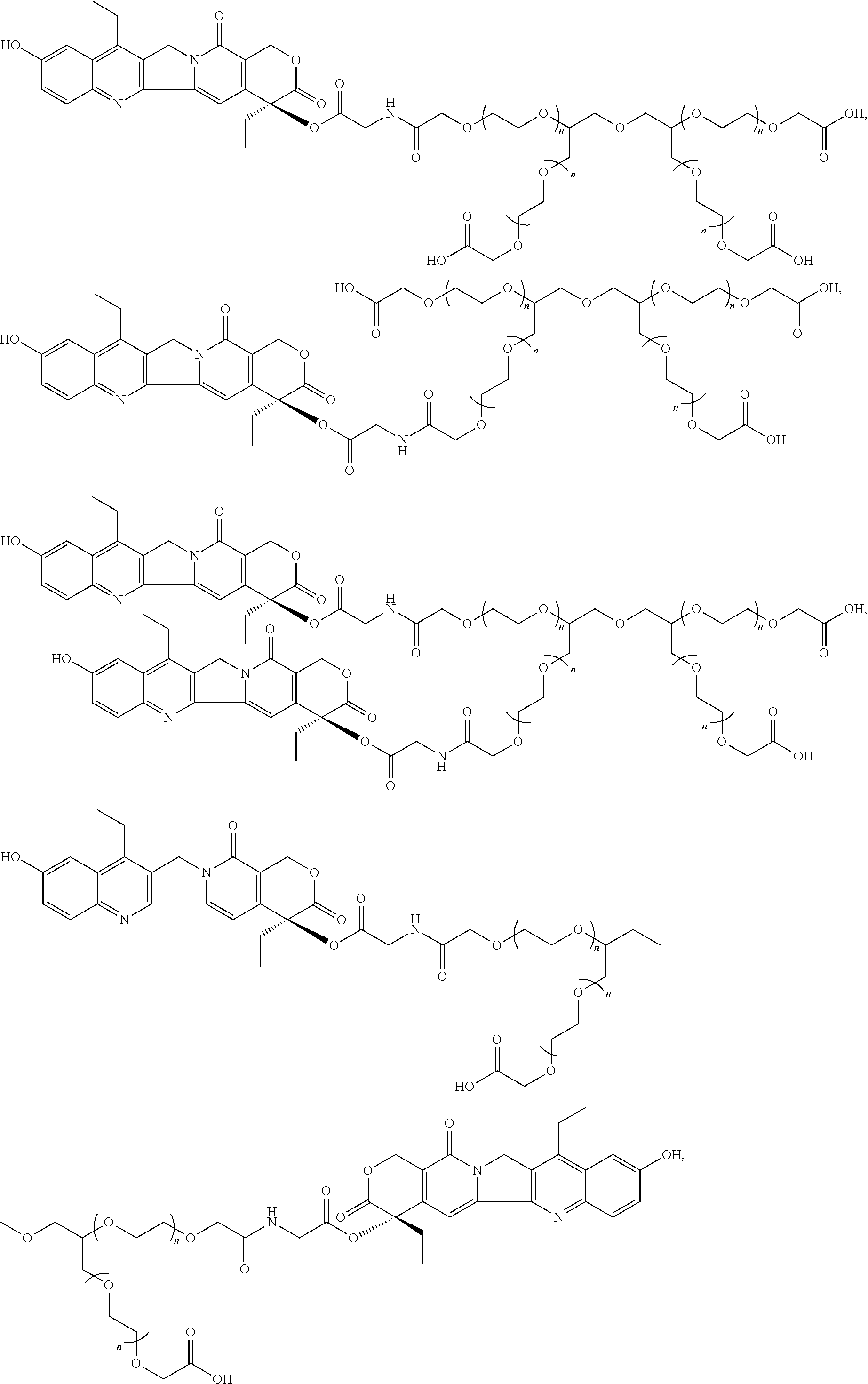

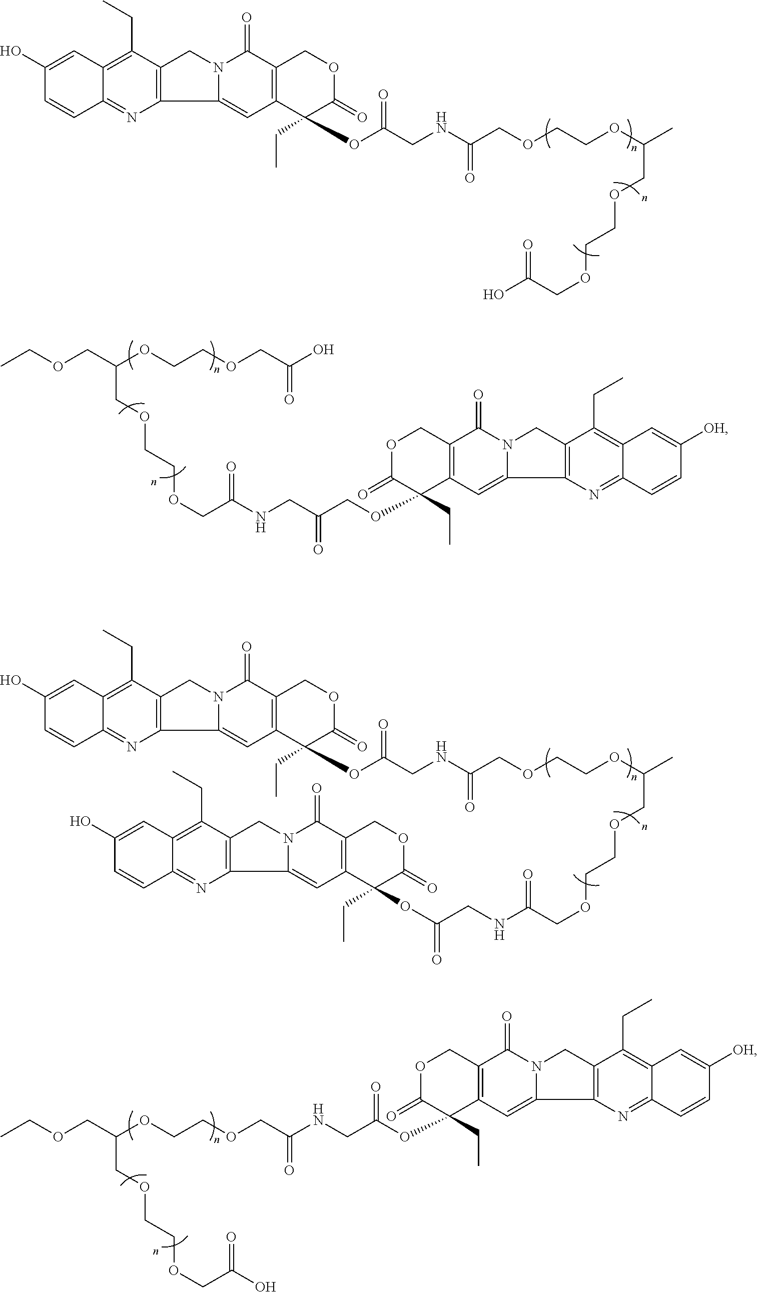

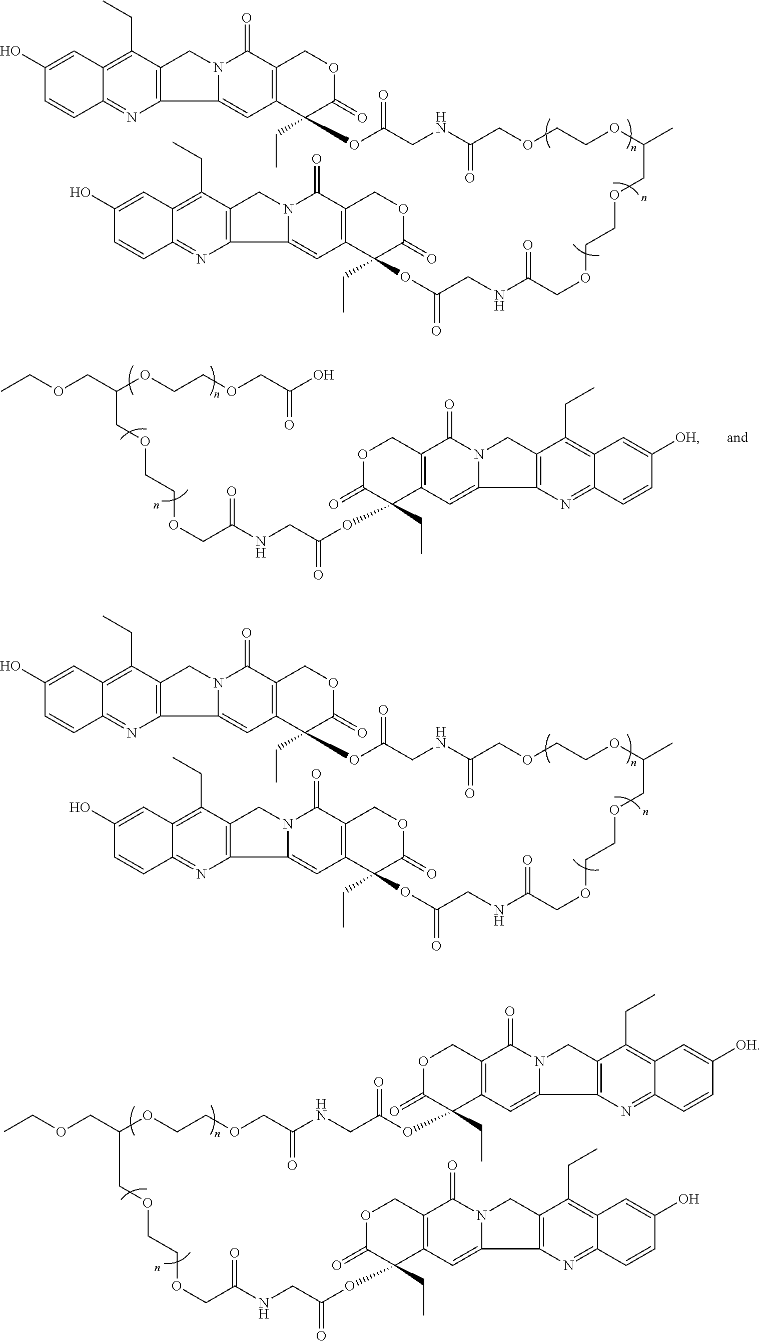

11. The method of claim 1, wherein the compound of Formula (I) is

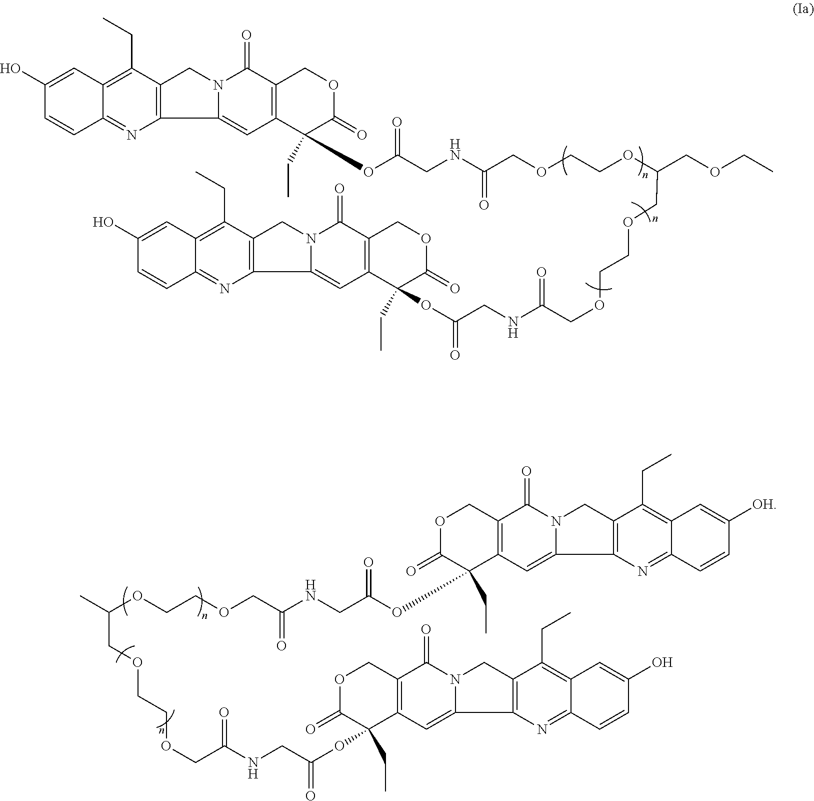

12. The method of claim 1, wherein the compound of Formula (I) is administered to the mammal, in amounts of from about 0.5 mg/m² body surface/dose to about 50 mg/m² body surface/dose, and wherein the amount is the weight of 7-ethyl-10-hydroxycamptothecin included in the compound of Formula (I).

13. The method of claim 1, wherein the compound of Formula (I) is administered to the mammal, in amounts of from about 1 mg/m² body surface/dose to about 18 mg/m² body surface/dose, and the amount is the weight of 7-ethyl-10-hydroxycamptothecin included in the compound of Formula (I).

14. The method of claim 1, wherein the compound of Formula (I) is administered to the mammal, according to a protocol of from about 1.25 mg/m² body surface/dose to about 16.5 mg/m² body surface/dose given weekly for three weeks, followed by 1 week without treatment, and the amount is the weight of 7-ethyl-10-hydroxycamptothecin included in the compound of Formula (I).

15. The method of claim 14, wherein the amount administered to the mammal weekly, is about 5 mg/m² body surface/dose, and the amount is the weight of 7-ethyl-10-hydroxycamptothecin included in the compound of Formula (I).

16. The method of claim 4, wherein the anti-HER2 receptor antibody is administered to the mammal, in an amount of from about 2 mg/kg to about 8 mg/kg.

17. The method of claim 2, wherein the antisense ErbB2 oligonucleotide or a pharmaceutically acceptable salt thereof is administered to the mammal, in combination with the compound of Formula (I) or an pharmaceutically acceptable salt thereof.

18. The method of claim 17, wherein the antisense ErbB2 oligonucleotide is complementary to at least 8 consecutive nucleotides of ErbB2 pre-mRNA or mRNA.

19. The method of claim 17, wherein the antisense ErbB2 oligonucleotide comprises from about 8 to 50 nucleotides in length.

20. The method of claim 17, wherein the antisense ErbB2 oligonucleotide comprises nucleotides that are complementary to at least 8 consecutive nucleotides set forth in SEQ ID NO: 1.

21. The method of claim 17, wherein the antisense ErbB2 oligonucleotide comprises one or more phophorothioate internucleotide linkages.

22. The method of claim 17, wherein the antisense ErbB2 oligonucleotide includes one or more locked nucleic acids (LNA).

23. The method of claim 17, wherein the antisense ErbB2 oligonucleotide is administered in an amount of from about 2 to about 50 mg/kg/dose.

24. The method of claim 1, further comprising determining the presence of a HER2 HER2 positive cancer in the mammal.

25. A method of treating a HER2 positive cancer in a mammal, comprising:
(a) identifying a mammal having a HER2 positive cancer by determining the presence, in the mammal, of a cancer that overexpresses HER2; and
(b) administering, to the mammal, an effective amount of a HER2 receptor antagonist comprising trastuzumab in combination with an effective amount of a compound of Formula (Ia): 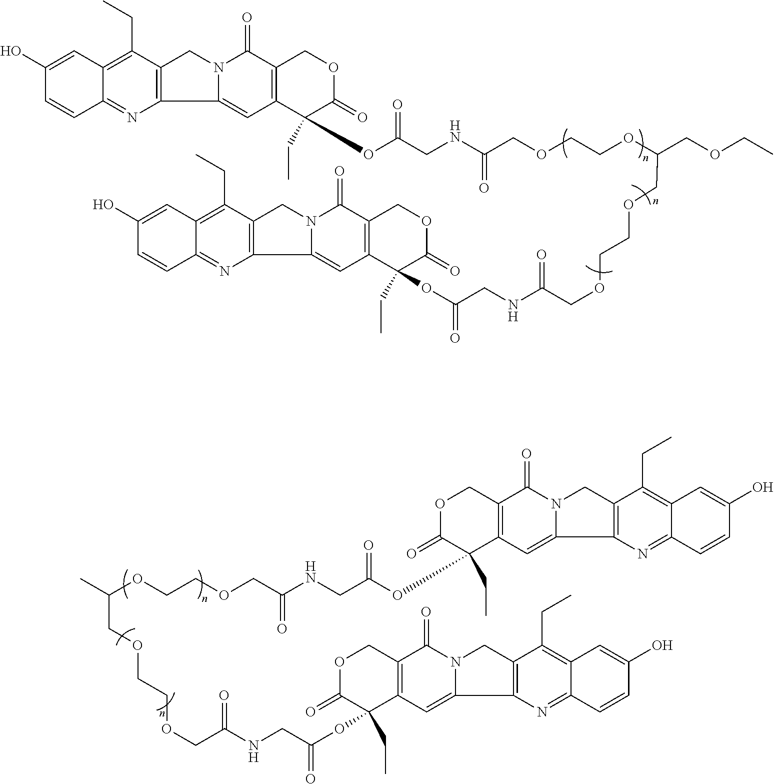
or a pharmaceutically acceptable salt thereof to the mammal having a HER2 positive cancer,
wherein (n) is about 227 so that the total molecular weight of the polymeric portion of the compound of Formula (Ia) is about 40,000 daltons.

26. A method of increasing HER2 receptor antagonist effects in a mammal having a HER2 positive cancer, comprising administering, to the mammal, a HER2 receptor antagonist in combination with an effective amount of a compound of Formula (I) of claim 1 or a pharmaceutically acceptable salt thereof.

27. A method of inhibiting the growth or proliferation of HER2 positive cells in a mammal, comprising
(a) determining the presence of a HER2 expression in cells in a mammal; and
(b) administering a HER2 receptor antagonist, to the mammal, in combination with a compound of Formula (I) of claim 1 or a pharmaceutically acceptable salt thereof to a mammal having HER2 positive cells.

28. A method of treating a HER2 positive cancer in a mammal, comprising administering to said mammal a HER2 receptor antagonist in combination with an effective amount of a camptothecin, a camptothecin analog, a polymeric conjugate of a camptothecin or analog thereof, or a pharmaceutically acceptable salt thereof.

29. The method of claim 28, wherein the polymeric conjugate is a compound of Formula (II) or Formula (III): 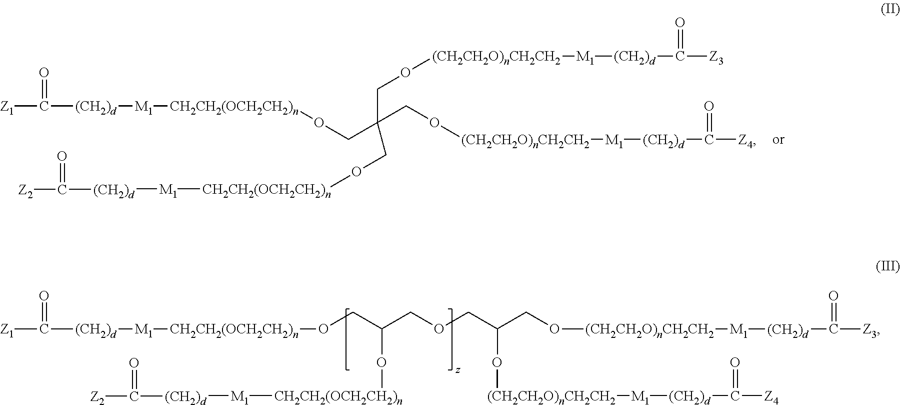
wherein
Z₁, Z₂, Z₃ and Z₄ are independently OH or (L)ₘ-D;
L is a bifunctional linker;
D is a camptothecin or a camptothecin analog;
M₁ is O, S, or NH;
(d) is zero or a positive integer of from about 1 to about 10;
(z) is zero or a positive integer of from 1 to about 29;
(m) is 0 or a positive integer; and
(n) is a positive integer of from about 10 to about 2,300 so that the polymeric portion of the compound has the total average molecular weight of from about 2,000 to about 100,000 daltons,
provided that Z₁, Z₂, Z₃ and Z₄ are not all OH.

30. The method of claim 29, wherein D is selected from the group consisting of camptothecin, SN38, topotecan, and CPT-11.

31. The method of claim 29, wherein the polymeric conjugate is 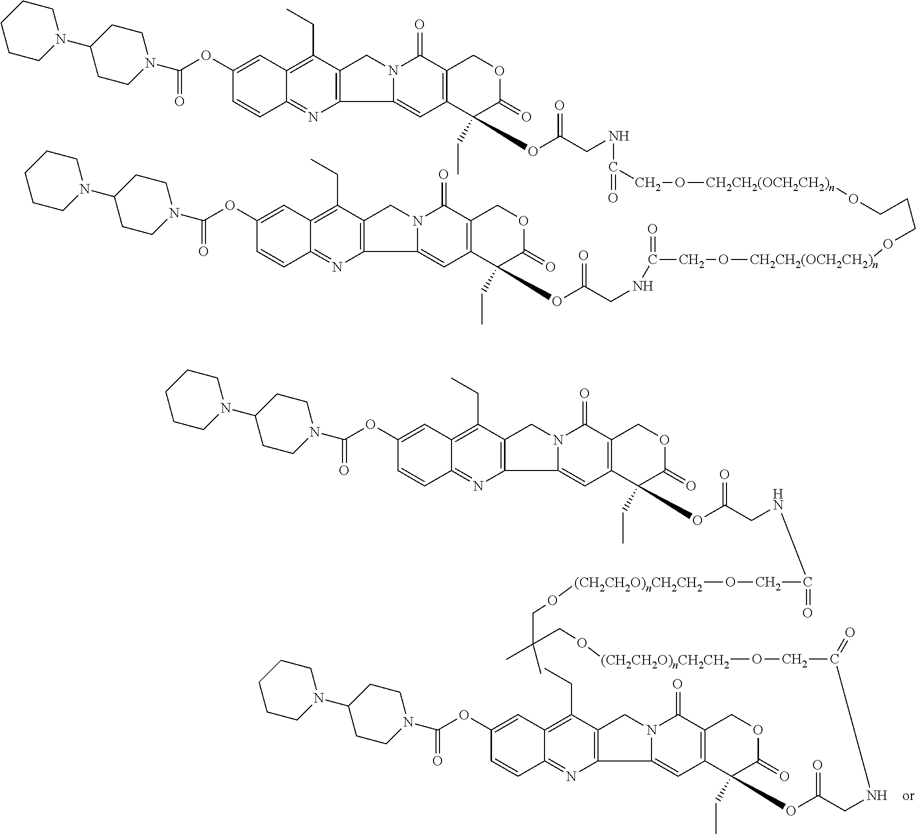 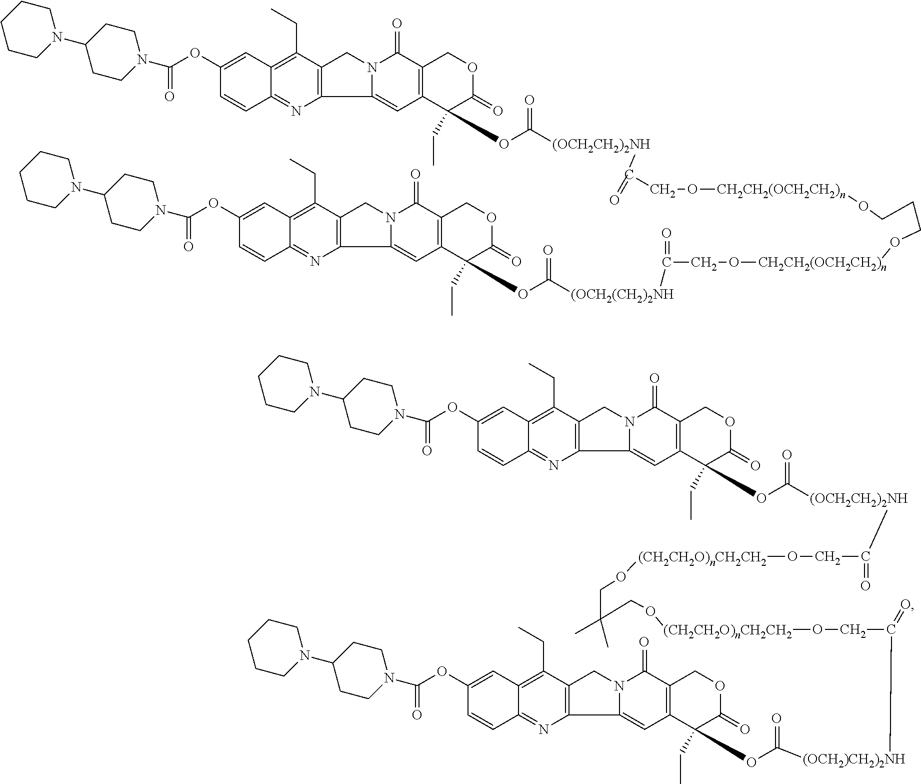
wherein (n) is an integer of from about 28 to about 341, so that the total molecular weight of the polymeric portion of the compound of Formula (II) ranges from about 5,000 to about 60,000 daltons.
